(19) United States
(12) Patent Application Publication
(10) Pub. No.: US 2011/0211991 A1
(43) Pub. Date: Sep. 1, 2011

(54) PROCESS CHALLENGE DEVICE AND METHOD

(76) Inventors: William E. Foltz, Cottage Grove, MN (US); Chad M. Herrlein, Woodbury, MN (US); Joshua M. Schmitz, Flemington, NJ (US); Barry W. Robole, St. Paul, MN (US); Kelvin J. Witcher, Hudson, WI (US); Paul N. Holt, Hudson, WI (US)

(21) Appl. No.: 13/128,093

(22) PCT Filed: Nov. 5, 2009
(86) PCT No.: PCT/US09/63416

§ 371 Date: May 6, 2011

Related U.S. Application Data
(60) Provisional application No. 61/112,071, filed on Nov. 6, 2008.
(60) Provisional application No. 61/112,149, filed on Nov. 6, 2008.
(60) Provisional application No. 61/112,041, filed on Nov. 6, 2008.

(51) Int. Cl. A61L 2/28; A61L 2/16

(52) U.S. Cl. 422/28; 422/119

(57) ABSTRACT

A sterilization process challenge device comprising a first container comprising walls which define a first space within the first container; a process indicator within the first space; at least one sterilant access for a sterilant to enter the first space within the first container; wherein at least one of the walls which defines the first space is a flexible wall; and wherein the walls are impervious to the sterilant; a method of using the device; and a kit including the device are disclosed.

### CROSS REFERENCE TO RELATED APPLICATIONS

This application claims the benefit of U.S. Provisional Application No. 61/112,041, filed Nov. 6, 2008, U.S. Provisional Application No. 61/112,149, filed Nov. 6, 2008, and U.S. Provisional Application No. 61/112,071, filed Nov. 6, 2008, each of which is incorporated herein by reference in its entirety.

### BACKGROUND

A variety of products and articles, including medical instruments, must be sterilized prior to use to prevent bio-contamination of a sample, an organism, a wound site, or the like. A number of sterilization processes are used which involve contacting the product or article with a fluid sterilant, such as a gaseous sterilant. Examples of such sterilants include, for example, steam, ethylene oxide, hydrogen peroxide, and the like.

The products and articles are generally packaged such that the sterilant can pass through the packaging, but microorganisms cannot pass through. Even though the sterilant can pass, the packaging restricts the movement of the sterilant to the product or article. Moreover, some products and articles include spaces within them that can only be reached by the sterilant via a restricted path. For example, endoscopes often include a long, narrow channel through which the sterilant must pass in order to sterilize the endoscope. These and other forms of restrictions associated with products and articles to be sterilized must be taken into account when employing a sterilization process, so that all surfaces of the product or article are exposed to the sterilant for a time sufficient to cause sterilization.

Monitoring for sufficient sterilization is generally carried out by placing an appropriate sterilization indicator along with the product and/or article to be sterilized within a sterilization chamber. A variety of sterilization indicators, including biological and chemical indicators, are known and used for this purpose. However, to take into account the above described restrictions encountered in the various products and articles, the sterilization indicator has been placed in a challenge device which restricts the flow of sterilant to the indicator using a long tortuous path. While such devices have been useful, they have not always been convenient to use and/or they have not always provided a close correlation between an indication of complete sterilization and actual complete sterilization of the product or article.

As such, there continues to be an interest in and a need for challenge devices which are convenient to use and provide a more reliable correlation between the indication of complete sterilization and actual complete sterilization of a product or article.

### SUMMARY OF THE INVENTION

In one embodiment, the present invention provides a process challenge device comprising:

a first container comprising walls which define a first space within the first container;

a process indicator within the first space; at least one sterilant access for a sterilant to enter the first space within the first container;

wherein at least one of the walls which defines the first space is a flexible wall; and wherein the walls are impervious to the sterilant.

The flexible wall allows the space within the container to change in volume as the pressure outside of the container changes. For example, when the pressure outside of the space is lowered, for example, when applying a vacuum, the volume may increase. In another example, when the pressure outside of the space is raised, for example, when applying a sterilant under pressure, the volume may decrease.

For certain embodiments, the process challenge device further comprises a heat-transfer modulating body adjacent the indicator. The heat-transfer modulating body may slow the rate at which the indicator comes to the temperature of a given sterilization process. For certain embodiments, the heat-transfer modulating body may also increase the time required for the sterilant to contact the indicator sufficiently to bring about an indication that sterilzation conditions have been achieved.

For certain embodiments, the sterilant access can be one or more openings, one or more ducts, one or more pressure-actuating valves, and a combination thereof. For certain embodiments, the sterilant access is sealed with a seal which can be removed when the device is put into use. For certain embodiments, at least two sterilant accesses are included. For certain embodiments, each of these sterilant accesses may be sealed with a removable seal. One, a portion, or all of the seals may be removed to prepare the device for use in a selected sterilization process.

For certain embodiments, the device further includes a second container, wherein the first container is within the second container. The second container comprises walls which define a second space within the second container; and at least one sterilant access for a sterilant to enter the second space; wherein the first container is within the second space; wherein at least one of the walls which defines the second space is a flexible wall; and wherein the walls which define the second space are impervious to the sterilant. Any of the sterilant access and volume features described herein for the first container may be included in the second container. For certain embodiments, this device provides the option of using the device with both first and second containers for a selected sterilization process, or removing the second container and using the resulting first container for another selected sterilization process. For certain embodiment, the first container may include removable seals on at least one but not all sterilant accesses included in the first container.

For certain embodiments, the device further includes at least one additional container, wherein the second container is within the at least one additional container. The at least one additional container comprises walls which define at least one additional space within the at least one additional container; and at least one sterilant access for a sterilant to enter the at least one additional space; wherein the second container is within the at least one additional space; wherein at least one of the walls which defines the at least one additional space is a flexible wall; and wherein the walls which define the at least one additional space are impervious to the sterilant. Any of the sterilant access and volume features described herein for the first container may be included in the at least one additional container. For certain embodiments, this device provides the option of using the device with both first, second, and the at least one additional containers for a selected sterilization process, or removing the at least one additional container and using the resulting device for another selected sterilization process. For certain embodiment, the second container may include removable seals on at least one but not all sterilant accesses included in the second container. For certain embodiments, the second container may also be removed and the resulting device used for another sterilization process as described above.

In another embodiment, there is provided a method of determining the effectiveness of a sterilization process, the method comprising:

providing a process challenge device, including any one of the embodiments thereof described herein;

positioning the process challenge device in a sterilization chamber;

exposing the process challenge device to a sterilant at an elevated temperature; and

determining whether or not the process indicator indicates that it has been exposed to sterilization process conditions effective for sterilizing an article.

For those embodiments wherein the sterilant accesses of the process challenge device are sealed with a removable seal, the above method further comprises removing at least one of the seals.

In another embodiment, there is provided a kit comprising a plurality of process challenge devices of any one of the embodiments of the process challenge device described herein.

In another embodiment, there is provided a kit comprising a plurality of process challenge devices selected from a plurality of the embodiments of the process challenge devices described herein. In one embodiment, for example, the plurality of process challenge devices may include different heat-transfer modulating bodies, different space volumes, different sterilant accesses, different quantities of sterilant accesses, different number of containers, or combinations thereof.

The above summary of the present invention is not intended to describe each disclosed embodiment or every implementation of the present invention. The description that follows more particularly exemplifies illustrative embodiments.

### DEFINITIONS

The terms “flexible wall” or “flexible walls” refers to a wall or walls which can be sufficiently deformed to allow at least a 5 percent, preferably at least a 10 percent, change in volume of a space defined by the wall or walls. The change in volume may result from a change in pressure outside of the space defined by the wall.

The term “heat-transfer modulating body” refers to a body which controls the time required to raise the temperature of an indicator adjacent the body to the sterilization process temperature. For example, where a steam sterilization process temperature is 132° C., the heat-transfer modulating body increases the time required for the indicator to reach 132° C. by slowing the rate at which heat is transferred to the indicator from, for example, a sterilization chamber.

The term “surround” refers to a heat-transfer modulating body or walls of the body positioned at least partially around the indicator but not completely enclosing the indicator.

The terms “envelop” or “enveloping” refer to a heat-transfer modulating body or walls of the body positioned to completely enclose the indicator.

The term “impervious to the sterilant” refers to walls that do not allow sterilant to pass through, except where an opening is provided to allow sterilant to enter any space defined by the walls. For example, the walls may be comprised of a continuous material which is not porous to the sterilant.

The term “pervious to the sterilant” refers to a heat-transfer modulating body or a wall or walls of the body that allow sterilant to pass through the body or the walls. For example, the body or walls may be comprised of a material which is porous to the sterilant, and/or the body or walls may include a plurality of openings or spaces through which the sterilant may pass.

The term “comprising” and variations thereof (e.g., comprises, includes, etc.) do not have a limiting meaning where these terms appear in the description and claims.

As used herein, “a,” “an,” “the,” “at least one,” and “one or more” are used interchangeably, unless the context clearly dictates otherwise.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Also herein, the recitations of numerical ranges by endpoints include all numbers subsumed within that range (e.g., a volume of 5 to 1000 cm³ includes a volume of 5, 63, 75.5, 1000 cm³ etc.).

### BRIEF DESCRIPTIONS OF THE DRAWINGS

FIG. 1 is a perspective view of one embodiment of a device according to the present invention, which includes an opening for the sterilant access.

FIG. 2 is a perspective view of another embodiment of a device according to the present invention, which includes a heat-transfer modulating body and a duct for a sterilant access.

FIG. 3 is a perspective view of one embodiment of a duct for a sterilant access.

FIG. 4 is a perspective view of another embodiment of a duct for a sterilant access.

FIG. 5 is a perspective view of another embodiment of a duct for a sterilant access.

FIG. 6 is a perspective view of another embodiment of a device according to the present invention, which includes a heat-transfer modulating body and an opening for a sterilant access.

FIG. 7A is a perspective view of another embodiment of a device according to the present invention, which includes a heat-transfer modulating body, an opening for a sterilant access, and a duct in fluid communication with the opening.

FIG. 7B is a partial schematic cross-section of the device of FIG. 7A further illustrating the opening and the duct.

FIG. 8 is a perspective view of another embodiment of a device according to the present invention, which includes a heat-transfer modulating body and an opening for a sterilant access.

FIG. 9A is a perspective view of another embodiment of a device according to the present invention, which includes a heat-transfer modulating body, a first container within a second container, and openings for a sterilant access in the first and second containers.

FIG. 9B is a schematic cross-section view of the device of FIG. 9A.

FIG. 10 is a perspective view of another embodiment of a device according to the present invention, which includes a heat-transfer modulating body with walls impervious to a sterilant and an opening for a sterilant access.

FIG. 11 is a perspective view of another embodiment of a device according to the present invention, which includes a heat-transfer modulating body and a valve for a sterilant access.

FIG. 12 is a perspective view of another embodiment of a device according to the present invention, which includes a heat-transfer modulating body and two valves for a sterilant access and for exiting a gas from the device, respectively.

FIG. 13 is a perspective view of the two valves in FIG. 12.

FIG. 14 is a perspective view of another embodiment of a device according to the present invention, which includes a heat-transfer modulating body, an opening for a sterilant access with a removable seal to seal the opening, and a duct for a second sterilant access.

FIG. 15 is a perspective view of another embodiment of a device according to the present invention, which includes a process indicator and a duct for a sterilant access.

FIG. 16 is a perspective view of another embodiment of a device according to the present invention, which includes a heat-transfer modulating body and two openings for sterilant accesses.

FIG. 17 is a perspective view of another embodiment of a device according to the present invention, which includes a heat-transfer modulating body, an opening for a sterilant access, and an absorbent sheet.

FIG. 18 is a perspective view of another embodiment of a device according to the present invention, which includes a heat-transfer modulating body, an opening for a sterilant access covered with a permeable vent material, and an absorbent sheet.

### DETAILED DESCRIPTION OF ILLUSTRATIVE EMBODIMENTS OF THE INVENTION

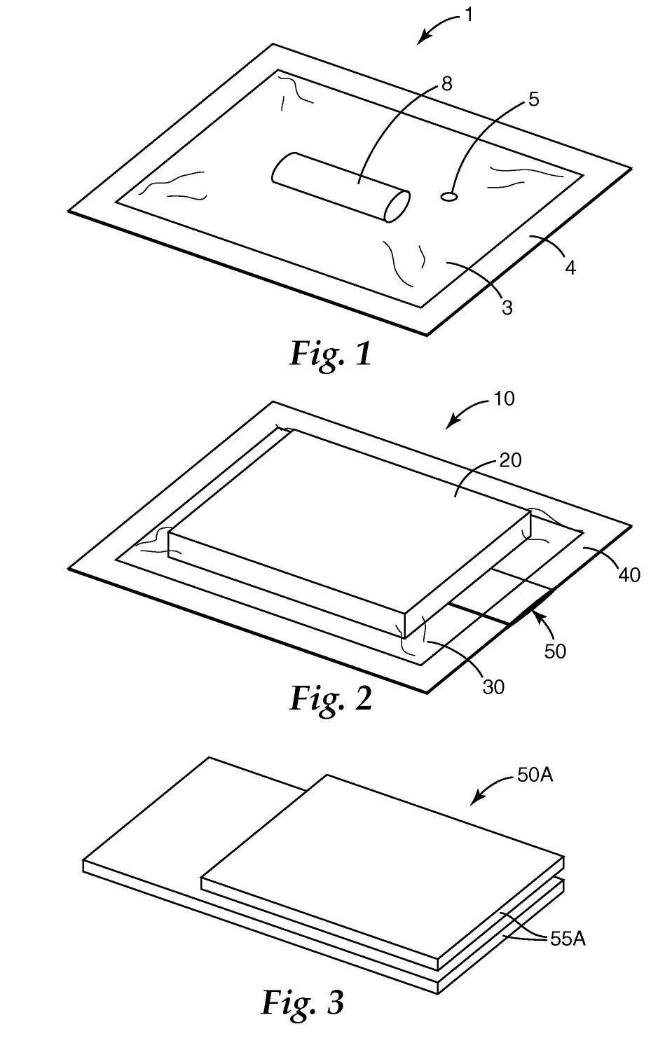

Process challenge device 1 illustrated in FIG. 1 is one embodiment of a process challenge device described herein. Container 3 is constructed with two sheets, each a flexible material and each forming a wall, heat sealed together at seal 4 to define the space in which process indicator 8 resides. For certain embodiments, the space within container 3 contains a volume of gas, typically determined at atmospheric pressure, of at least 5 cubic centimeters. For certain embodiments, the volume of gas is not more than 100 cubic centimeters. Although not shown, additional sheets could be used to form additional walls, for example, one or more gussets. Additional sheets could also be used together with the two sheets to add additional layers to the walls. Examples of suitable flexible materials include films of nylon, polyester, polyethylene, polypropylene, foil, and the like, and combinations thereof. For certain embodiments, the films are laminate films, examples of which include nylon/polyethylene/foil/polyethylene laminate films, clear polyester/polypropylene laminate films, and the like.

For certain embodiments, including any one of the device, method, and kit embodiments described herein, all of the walls of the first container are flexible walls.

For certain embodiments, including any one of the device, method, and kit embodiments described herein which include a second container, all of the walls of the second container are flexible walls.

For certain embodiments, including any one of the device, method, and kit embodiments described herein which include at least one additional container, all of the walls of the at least one additional container are flexible walls.

For certain embodiments, including any one of the device, method, and kit embodiments described herein, the flexible walls are laminate film.

Device 1 also includes opening 5 as a sterilant access in one of the two walls. For certain embodiments, the area of opening 5 is at least 0.1, 0.2, or 0.5 mm², and for certain embodiments, opening 5 has an area of not more than 100, 50, 20, or 10 cm². The size of opening 5 can be selected for a selected sterilization process. For example, for a sterilization process which includes vacuum cycles, the opening 5 may be relatively small. For certain embodiments, the area of opening 5 is at least 0.5 mm² and not more than 20 cm². In another example, for a gravity sterilization process without vacuum cycles, the opening 5 may be relatively large. For certain embodiments, the area of opening 5 is at least 10 cm² and not more than 100 cm². Device 1 is shown with one opening 5. However, more than one opening can be included. The openings can each have the same area or different areas. Some or all of the openings can be sealed with a removable seal, and one or more of the seals can selected and removed for using device 1 in a selected sterilization process.

Device 1 is illustrated with both walls being a flexible material, although in other embodiments one or more of the walls can be a rigid material as long as at least one wall is flexible. Because at least one wall of device 1 is flexible, relatively thin and low cost materials can be used for the wall or walls. Moreover, in certain embodiments, the flexible material allows the volume of the space defined by the walls to vary as a pressure differential varies between the space and outside the space or container. Thus, when the pressure outside of the container is increase, such as when a sterilant under pressure is applied to the container, the volume of the space decreases. This may increase the resistance of the device to a sterilant by resisting or slowing entrance of the sterilant into the space. Furthermore, when the pressure outside of the container is reduced, such as when applying a vacuum to the device to remove air or another gas from the space to facilitate displacement of the air or gas by the sterilant, the volume of the container increases. This may increase the resistance of the device as well by resisting or slowing exiting of the air or gas from the space.

Process indicator 8 or any process indicator referred to in any of the embodiments described herein can be one or more indicators and one or more types of indicators, for example, a biological indicator (BI) and/or a chemical indicator (CI).

Process challenge device 10 illustrated in FIG. 2 is another embodiment of a process challenge device described herein. Device 10 includes container 30 constructed with two sheets, each a flexible material and each forming a wall, heat sealed together at seal 40 to define the space in which heat-transfer modulating body 20 resides. Heat-transfer modulating body 20 envelops a process indicator such as described in FIG. 1 for indicator 8. Because heat-transfer modulating body 20 envelops the indicator, at least a portion of the body or at least a portion of walls comprising the body are porous. In one example, the body 20 is an ATTEST Rapid 5 Test Pack Plus #41382 (available form 3M Company, St. Paul, Minn.) This envelops an ATTEST 1292 Rapid Biological Indicator and COMPLY STERIGAGE 1243 Steam Chemical Integrator (all available from 3M Company, St. Paul, Minn.). For certain embodiments, the space within container 30 contains a volume of gas, typically determined at atmospheric pressure, of at least 5 cubic centimeters. For certain embodiments, the volume of gas is not more than 100 cubic centimeters. Although not shown, additional sheets could be used to form additional walls, for example, one or more gussets. Additional sheets could also be used together with the two sheets to add additional layers to the walls. Examples of suitable sheet material are described above in reference to FIG. 1.

Device 10 also includes duct 50 as a sterilant access entering through seal 40 and extending to heat-transfer modulating body 20. Duct 50 comprises at least two layers of sheet material, each having an area and at least one major surface adjacent to a major surface of another of the at least two layers, each major surface comprising at least a portion of the area. FIGS. 3, 4, and 5 illustrate some embodiments of duct 50 that may be used in device 10 or any of the embodiments of a device described herein which include a duct. Duct 50A illustrated in FIG. 3 includes two layers of sheet material 55A, each having a major surface adjacent to a major surface of the other layer. Any sterilant entering the space within container 30 or any gas and/or liquid exiting the space travels through the area where the major surfaces are adjacent each other. For certain embodiments, the major surfaces adjacent to each other have an area of at least 10 mm² where the major surfaces are adjacent to each other. For certain embodiments, the area is at least 10 cm². For certain embodiments, the major surfaces adjacent to each other have an area of not more than 100 cm² where the major surfaces are adjacent to each other.

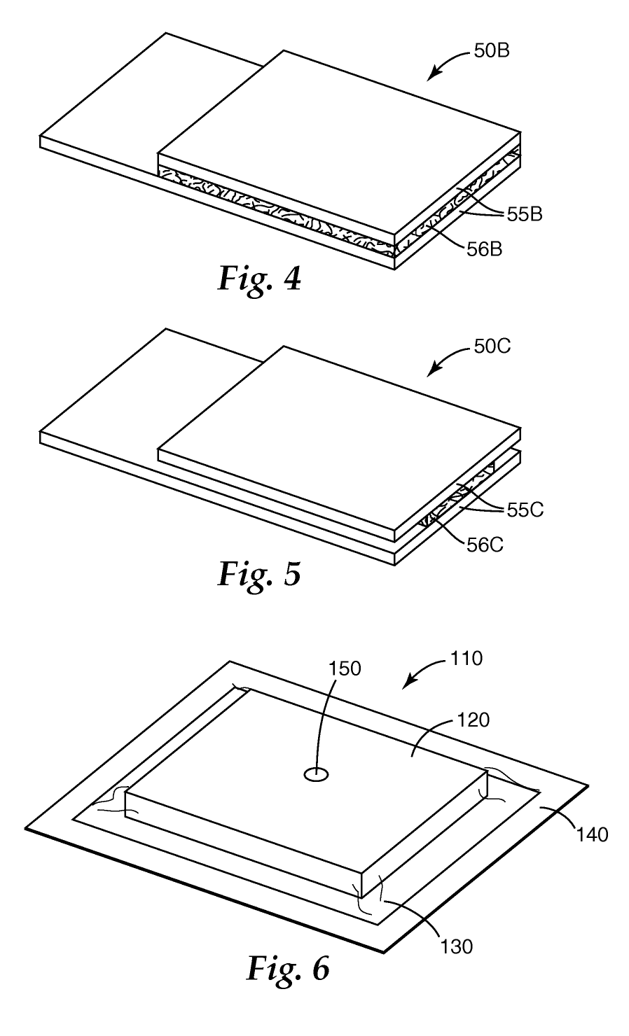

Duct 50B illustrated in FIG. 4 further includes a porous spacer 56B between the layers of sheet material 55B. Examples of the porous spacer include, for example, woven, knit, and nonwoven fabrics or mats, particles or beads held in place on the adjacent surfaces of the sheet material or on an additional sheet material, protrusions on the surfaces of the sheet material or on an additional sheet material, and the like. For certain embodiments, the porous spacer is removable. For certain embodiments, the porous spacer is a nonwoven fabric or mat. The presence of porous spacer 56B may allow sterilant to pass through duct 50B more easily than through duct 50A shown in FIG. 3, and thereby reduce the resistance of a device for use in a selected a sterilization process.

Duct 50C illustrated in FIG. 5 includes a porous spacer 56C which is narrower than the width of the layers of sheet material 55C. The amount by which porous spacer 56C is narrower than the width of the layers of sheet material 55C may further increase the ease of a sterilant to pass through duct 50C, and thereby reduce the resistance of a device for use in a selected sterilization process.

Process challenge device 110 illustrated in FIG. 6 is another embodiment of a process challenge device described herein. Device 110 includes container 130 constructed with two sheets, each a flexible material and each forming a wall, heat sealed together at seal 140 to define the space in which heat-transfer modulating body 120 resides. Heat-transfer modulating body 120 envelops a process indicator such as described in FIG. 1 for indicator 8 or as described in FIG. 2 for heat-transfer modulating body 20. For certain embodiments, the space within container 130 contains a volume of gas, typically determined at atmospheric pressure, of at least 5 cubic centimeters. For certain embodiments, the volume of gas is not more than 100 cubic centimeters. Although not shown, additional sheets could be used to form additional walls, for example, one or more gussets. Additional sheets could also be used together with the two sheets to add additional layers to the walls. Examples of suitable sheet material are described above in reference to FIG. 1.

Device 110 also includes opening 150 as a sterilant access in one of the two walls. For certain embodiments, the area of opening 150 is at least 0.5 mm², and for certain embodiments, opening 150 has an area of not more than 100 cm². The size of opening 150 can be selected for a selected sterilization process. For example, for a sterilization process which includes vacuum cycles, the opening 150 may be relatively small. For certain embodiments, the area of opening 150 is at least 0.5 mm² and not more than 20 cm². In another example, for a gravity sterilization process without vacuum cycles, the opening 150 may be relatively large. For certain embodiments, the area of opening 150 is at least 10 cm² and not more than 100 cm². Device 110 is shown with one opening 150. However, more than one opening can be included. The openings can each have the same area or different areas. Some or all of the openings can be sealed with a removable seal, and one or more of the seals can selected and removed for using device 110 in a selected sterilization process.

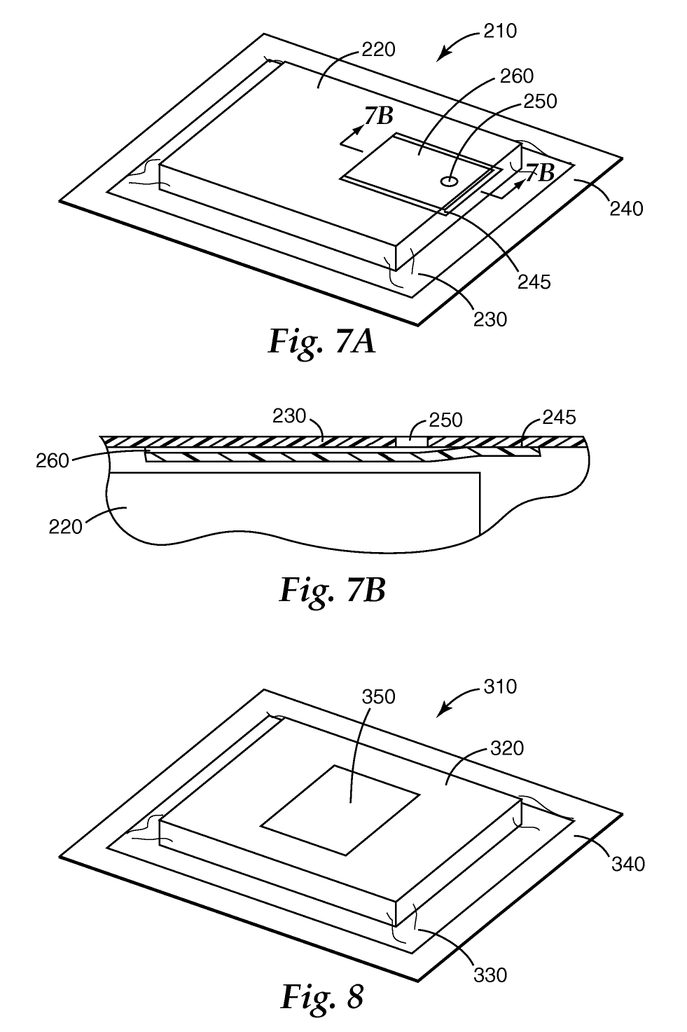

Process challenge device 210 illustrated in FIGS. 7A and 7B is another embodiment of a process challenge device described herein. Device 210 is similar to device 110 illustrated in FIG. 6 with container 230 constructed with two sheets, each a flexible material and each forming a wall, heat sealed together at seal 240 to define the space in which heat-transfer modulating body 220 resides, and with opening 250 as a sterilant access in one of the two walls. Device 210 further includes duct 260 through which a sterilant may enter container 230 after entering opening 250. Duct 260 is constructed by sealing at seals 245 a sheet material to the inside of the wall with the opening 250. Duct 260 may provide a more consistent resistance to entrance of a sterilant or exiting of a gas as the heat-transfer modulating body is modified or various body designs, materials, and surfaces are selected for a selected sterilization process.

Process challenge device 310 illustrated in FIG. 8 is another embodiment of a process challenge device described herein. Device 310 is similar to device 110 illustrated in FIG. 6 with container 330 constructed with two sheets, each a flexible material and each forming a wall, heat sealed together at seal 340 to define the space in which heat-transfer modulating body 320 resides. Opening 350 is a sterilant access in one of the two walls and is larger than the opening 150 illustrated in FIG. 6. Opening 350 may be selected for a selected process as discussed above for device 1. In one example, the selected process can be a gravity sterilization process which does not use a vacuum cycle. For certain embodiments, the area of opening 350 is at least 10 cm² and not more than 100 cm².

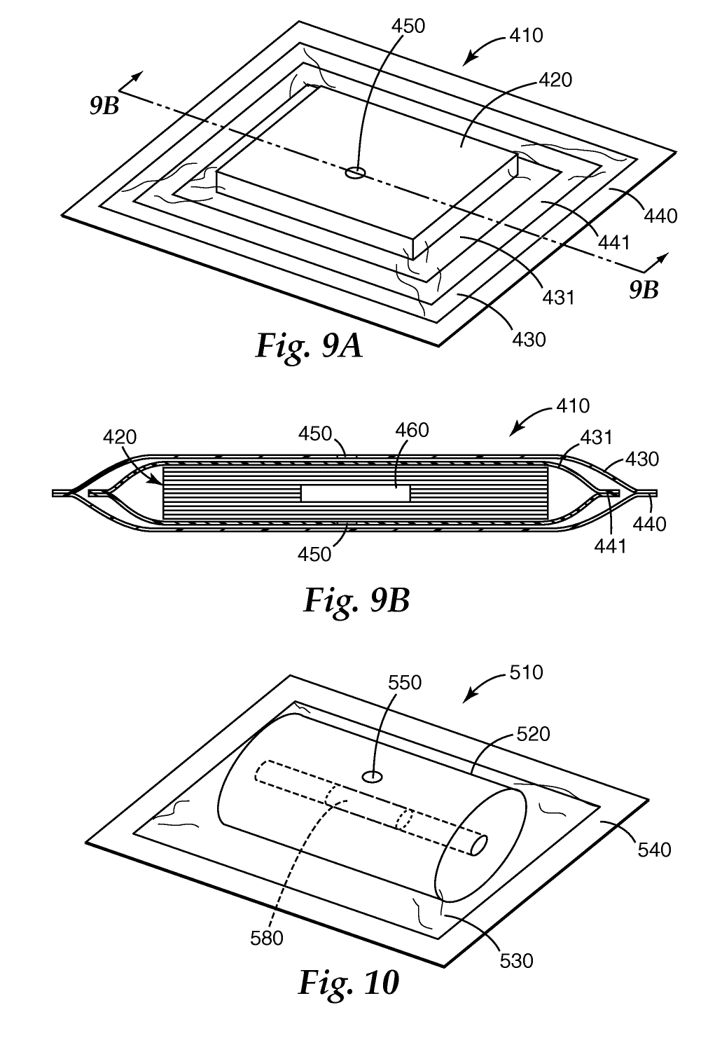

Process challenge device 410 illustrated in FIGS. 9A and 9B is another embodiment of a process challenge device described herein. Device 410 is similar to device 110 illustrated in FIG. 6 with first container 431 constructed with two sheets, each a flexible material and each forming a wall, heat sealed together at seal 441 to define the space in which heat-transfer modulating body 420 resides, and with opening 450 a sterilant access in one of the two walls. Device 410 further includes second container 430 also constructed with two sheets, each a flexible material and each forming a wall, heat sealed together at seal 440 to define a second space in which first container 431 resides. Second container 430 also includes opening 450 on the opposite side of opening 450 in first container 431 as illustrated in FIG. 9B. The cross-section of device 410 in FIG. 9B also illustrates a cross-section of heat-transfer modulating body 420 illustrating space 460 for a process indicator (not shown) and layers of material comprising walls of the heat-transfer modulating body which envelop space 460 and an indicator therein. The design of heat-transfer modulating body 420 illustrated in FIG. 9B may provide unexpected resistance to entrance of a sterilant into space 460 when used in the present flexible container, such as containers 430 and 431. When pressure is increased outside of container 430 and 431, such as when a sterilant under pressure is applied to the containers, the containers reduce in volume, compressing body 420, and thereby increasing the resistance of the device to a sterilant.

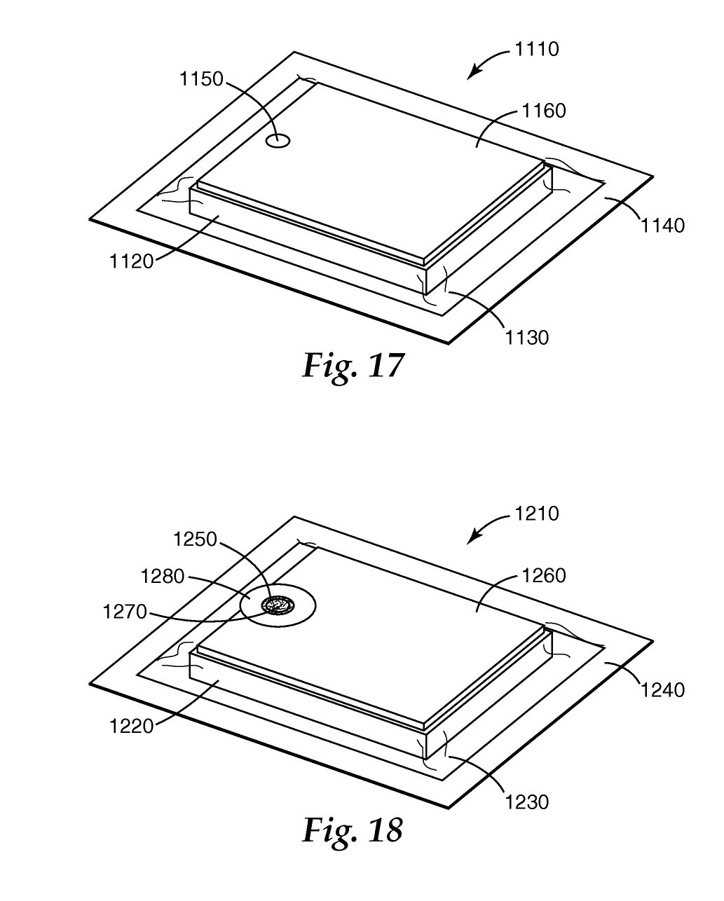

Although not shown, process challenge device 410 may include an absorbent material as described and illustrated below in reference to FIGS. 17 and 18. The absorbent material may be positioned between containers 430 and 431 and adjacent openings 450. The absorbent material may also be positioned between heat-transfer modulating body 420 and the wall of container 431 with opening 450.

Process challenge device 510 illustrated in FIG. 10 is another embodiment of a process challenge device described herein. Device 510 is similar to device 110 illustrated in FIG. 6 with first container 530 constructed with two sheets, each a flexible material and each forming a wall, heat sealed together at seal 540 to define the space in which heat-transfer modulating body 520 resides, and with opening 550 a sterilant access in one of the two walls. Heat-transfer modulating body 520, however, is a solid body which surrounds but does not envelop process indicator 580. Except for the opening in body 520 through which indicator 580 is inserted, body 520 may be impervious to a sterilant. Body 520 may be selected for a selected sterilization process with the walls of body 520 being thicker and/or having a lower thermal diffusivity for greater resistance or thinner and/or having a higher thermal diffusivity for lesser resistance to a sterilization process.

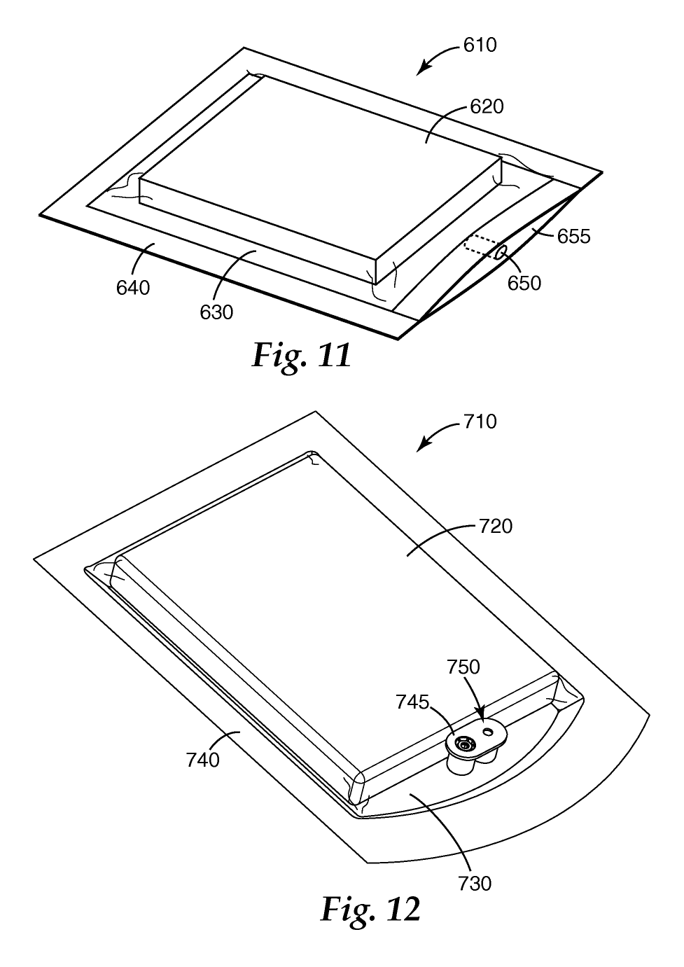

Process challenge device 610 illustrated in FIG. 11 is another embodiment of a process challenge device described herein. Device 610 is similar to device 110 illustrated in FIG. 6 with first container 630 constructed with two sheets, each a flexible material and each forming a wall, heat sealed together at seal 640 to define the space in which heat-transfer modulating body 620 resides. Device 610 includes pressure-actuating valve 650 as a sterilant access mounted in housing 655 sealed to the two sheets. Device 610 is illustrated with one valve, but more than one valve may be used. For certain embodiments, the at least one pressure-actuating valve is a combination valve. For certain embodiments, the at least one pressure-actuating valve is actuated when there is a pressure difference between space within the container and outside of the container. For certain embodiments, the pressure difference is at least 6.895 kPa (1 psi). For certain embodiments, the pressure difference is not more than 345 kPa (50 psi). For certain embodiments, the sterilant access comprises at least two pressure-actuating valves. For certain embodiments, at least one pressure-actuating valve is actuated when the pressure is higher outside of the container than in the space within the container. For certain embodiments, the pressure is higher by at least 6.895 kPa (1 psi). For certain embodiments, the pressure is higher by not more than 345 kPa (50 psi).

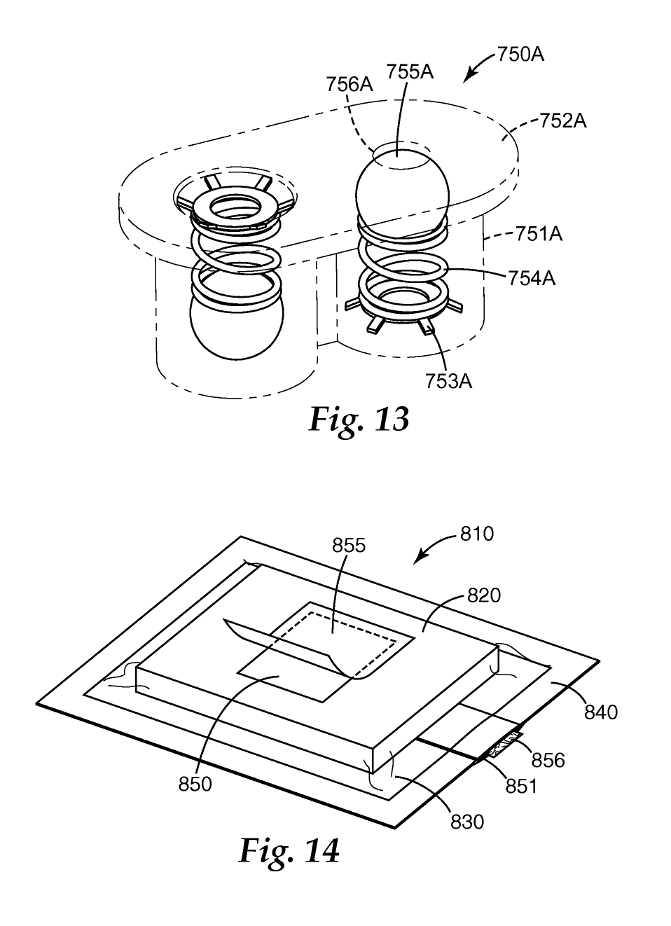

Process challenge device 710 illustrated in FIG. 12 is another embodiment of a process challenge device described herein. Device 710 is similar to device 110 illustrated in FIG. 6 with first container 730 constructed with two sheets, each a flexible material and each forming a wall, heat sealed together at seal 740 to define the space in which heat-transfer modulating body 720 resides. Device 710 includes pressure-actuating double valve 750 as a sterilant access sealed to one of the walls with seal 745. Valves 750 are positioned so that a sterilant enters the space within container 730 through one of the valves and a gas and/or liquid exits the space within container 730 through the other valve. Valve 750 is further illustrated in FIG. 13 as valve 750A. Seal 745 seals the sheet material of the wall to valve housing flange 752A. Ball 755A, spring 754A, and spring retainer 753A are positioned within valve housing 751A. A gas and/or liquid passes through opening 756A in valve housing 751A when the valve is actuated.

Process challenge device 810 illustrated in FIG. 14 is another embodiment of a process challenge device described herein. Device 810 is similar to both devices 310 illustrated in FIGS. 8 and 10 illustrated in FIG. 2. Container 830 is constructed with two sheets, each a flexible material and each forming a wall, heat sealed together at seal 840 to define the space in which heat-transfer modulating body 820 resides. Opening 850 is a sterilant access in one of the two walls and is sealed with a removable seal 855.

Device 810 also includes duct 851 as a sterilant access entering through seal 840 and extending to heat-transfer modulating body 820. Duct 851 comprises at least two layers of sheet material, each having an area and at least one major surface adjacent to a major surface of another of the at least two layers, each major surface comprising at least a portion of the area. Duct 851 is illustrated as duct 50C illustrated in FIG. 5 except that porous spacer 856 protrudes from duct 851 sufficiently for ease of removing porous spacer 856 for use in a selected sterilization process. Any of the embodiments of a duct described in reference to FIG. 2 may be used.

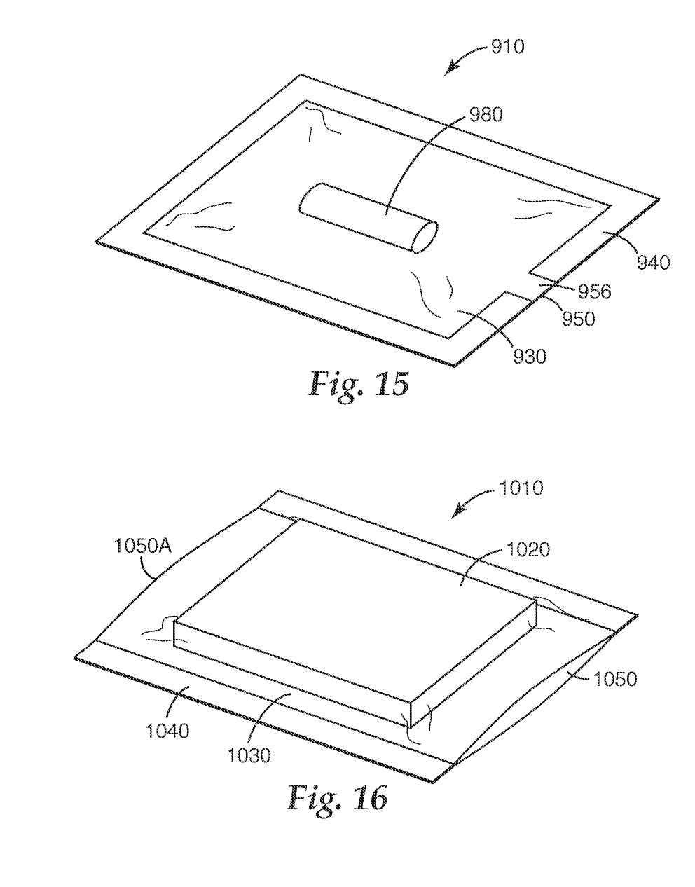

Process challenge device 910 illustrated in FIG. 15 is another embodiment of a process challenge device described herein. Device 910 is similar to device 1 illustrated in FIG. 1 with container 930 constructed with two sheets, each a flexible material and each forming a wall, heat sealed together at seal 940 to define the space in which process indicator 980 resides. Duct 950 is a sterilant access in an edge of container 930. Duct 950 is constructed by leaving sheet material portion 956 unsealed. Here the at least two layers of sheet material comprising duct 950 are an extension of the walls formed by the flexible material.

Process challenge device 1010 illustrated in FIG. 16 is another embodiment of a process challenge device described herein. Device 1010 includes container 1030 constructed with two sheets, each a flexible material and each forming a wall, heat sealed together at seal 1040 to define the space in which heat-transfer modulating body 1020 resides. Openings 1050 and 1050A are sterilant accesses. The area of openings 1050 and 1050A can be conveniently determined by not sealing the flexible material at each end of the device as illustrated or by sealing a portion of the flexible material at each end of the device.

Process challenge device 1110 illustrated in FIG. 17 is another embodiment of a process challenge device described herein. Device 1110 includes container 1130 constructed with two sheets, each a flexible material and each forming a wall, heat sealed together at seal 1140 to define the space in which heat-transfer modulating body 1120 resides. Heat-transfer modulating body 1120 envelops a process indicator as described above for FIG. 6. For certain embodiments, the space within container 1130 contains a volume of gas as described above for FIG. 6. Although not shown, additional sheets could be used to form additional walls, for example, one or more gussets. Additional sheets could also be used together with the two sheets to add additional layers to the walls. Examples of suitable flexible material are described above in reference to FIG. 1. Device 1110 also includes opening 1150 as a sterilant access in one of the two walls. For certain embodiments, the area of opening 1150 is as described above for FIG. 6. Device 1110 is shown with one opening 1150. However, more than one opening can be included. The openings can each have the same area or different areas. Some or all of the openings can be sealed with a removable seal, and one or more of the seals can selected and removed for using device 1110 in a selected sterilization process.

Device 1110 also includes absorbent material 1160 for absorbing a condensate, such as a condensate of a sterilant, for example, a condensate of steam. Absorbent material 1160 is shown as a sheet material adjacent opening 1150. However, forms other than sheets of absorbent material may be used. Examples of absorbent materials that may be used include any one of or a combination of fibers, webs, films, microporous films, membranes, absorbents, foams, powders, gums, polymeric gels, microparticles, nanoparticles, nonwoven materials, including spunbond materials, knitted materials, meltblown materials, and composite materials. Suitable materials include any one or a combination of cellulosic fibers; cotton; glass; rayon; nylon; synthetic fibers such as polyvinyl alcohol, polyvinyl chloride, polybutylene terephthalate, polytetrafluoroethylene, polypropylene, polyethylene, polylactic acid, polyester, and polyurethane; wood pulp; acrylics; olefin; wool; paper; metal; superabsorbent polymers; superabsorbent particles; cheesecloth; polymeric gels and hydrogels such as copolymers of polyvinylpyrrolidone and any of the polymeric gels disclosed in U.S. Pat. No. 6,352,837 (Witcher) which are incorporated herein by reference.

Process challenge device 1210 illustrated in FIG. 18 is another embodiment of a process challenge device described herein. Device 1210 includes container 1230 constructed with two sheets, each a flexible material and each forming a wall, heat sealed together at seal 1240 to define the space in which heat-transfer modulating body 1220 resides. Heat-transfer modulating body 1220 envelops a process indicator as described above for FIG. 6. For certain embodiments, the space within container 1230 contains a volume of gas as described above for FIG. 6. Although not shown, additional sheets could be used to form additional walls, for example, one or more gussets. Additional sheets could also be used together with the two sheets to add additional layers to the walls. Examples of suitable flexible material are described above in reference to FIG. 1.

Device 1210 also includes opening 1250 as a sterilant access in one of the two walls. For certain embodiments, the area of opening 1250 is as described above for FIG. 6. Device 1210 is shown with one opening 1250. However, more than one opening can be included. The openings can each have the same area or different areas. Some or all of the openings can be sealed with a removable seal, and one or more of the seals can selected and removed for using device 1210 in a selected sterilization process.

Device 1210 also includes absorbent material 1260 for absorbing a condensate, such as a condensate of a sterilant, for example, a condensate of steam. Absorbent material 1260 is shown as a sheet material adjacent opening 1250. Absorbent materials as described above for Device 1110 may be used.

Device 1210 also includes permeable vent material 1270 covering opening 1250. The vent material is permeable to the sterilant. Preferably, the vent material is also permeable to any gas, such as air, within container 1230. Suitable permeable vent materials include porous webs, microporous films, porous membranes, at least partially reticulated foams, woven materials, nonwoven materials, including spunbond materials, knitted materials, meltblown materials, and composite materials.

The permeable vent material increases resistance of the device to the sterilant, for example, by slowing the rate of sterilant entering the container. For certain embodiments, including any one of the embodiments described herein which includes an opening for a sterilant access, the opening is covered with a permeable vent material.

Device 1210 also includes stiffening material 1280. The stiffening material reduces or prevents wrinkling of the flexible material which makes up container 1230 at opening 1250, during a sterilization cycle. This maintains consistency of the opening size during a sterilization cycle, thereby increasing consistency of the challenge provided by the device. Stiffening material 1280 in device 1210 is attached to container 1230 by a pressure sensitive adhesive which also serves to retain permeable vent material 1270 in position covering opening 1250. Suitable stiffening materials include films, including polymeric films, metal sheeting, including foils, papers, card boards, polymer coatings, nonwovens, gels, and combinations thereof. The stiffening material may be attached to the area around the sterilant access opening by an adhesive, including a pressure sensitive adhesive, thermal bonding, or the like.

For certain embodiments, including any one of the embodiments described herein which includes an opening for a sterilant access, the opening is at least partially surrounded with a stiffening material. For certain of these embodiments, the opening is fully surrounded with stiffening material, for example, as illustrated by stiffening material 1280 in FIG. 18.

For certain embodiments, including any one of the embodiments described herein, an absorbent material which absorbs sterilant condensate is included within the process challenge device container. The absorbent material may be any one or combination of those described above. When more than one container is included in the device, for example, as shown in FIGS. 9A and 9B, the absorbent material may be included within any one of the containers, all of the containers, or a portion of the containers. For certain embodiments, preferably the absorbent material is positioned as described for FIG. 9B and as shown in FIGS. 17 and 18.

For certain embodiments, including any one of the embodiments described herein which includes an absorbent material, the absorbent material is a layer of absorbent material or sheet material. Sheet materials include nonwovens, knits, wovens, papers, card stock, cardboard, porous membranes, porous films, and the like. For certain of these embodiments, the sheet material is a nonwoven or a paper towel. For certain of these embodiments, preferably the layer of absorbent material or the sheet material has a thickness of at least 0.25 mm, preferably at least 0.4 mm, more preferably at least 1 mm. For certain of these embodiments, the layer of absorbent material or the sheet material has a thickness of not more than 10 mm, preferably not more than 7.5 mm, more preferably not more than 5.5 mm. For certain of these embodiments, the thickness is 1 mm to 7.5 mm. For certain of these embodiments, the thickness is 2 mm to 5.5 mm.

For certain embodiments, including any one of the embodiments described herein which includes an absorbent material, the absorbent material is positioned adjacent a sterilant access. The sterilant access may be an opening, a duct, a pressure-actuating valve, or a combination thereof. For certain of these embodiments, preferably the sterilant access is an opening. When positioned adjacent a sterilant access at least a portion of sterilant entering the container passes by and/or through the absorbent material. For certain of these embodiments, the absorbent material is positioned adjacent a sterilant access such that all of the sterilant entering the container passes by and/or through the absorbent material.

The layer of absorbent material or the absorbent sheet material covers a sufficient area and is present in sufficient amount to prevent sterilant condensate from sealing off the sterilant access in an uncontrolled or non-reproducible manner during a sterilization cycle.

Incorporation of the absorbent material adjacent a sterilant access, such as opening 1150 in FIG. 17 and opening 1250 in FIG. 18, has now been found to make the operation of the process challenge device more consistent and prevent inadvertent sealing of the opening from entrance of the sterilant, caused by formation of condensate within the container.

For certain embodiments, the walls comprising any one of the embodiments of a heat-transfer modulating body described herein have a thickness of at least 0.3 cm. The walls may include one, two, three, or more layers. The heat-transfer modulating body can be adjusted for wall thickness by removing one or more layers, and thereby decrease the resistance of the device to sterilization conditions. Also, one or more additional layers can be added to increase the wall thickness of the heat-transfer modulating body, and thereby increase the resistance of the device to sterilization conditions. Moreover, the layers can have the same or different thermal diffusivities, allowing the thermal diffusivity of the heat-transfer modulating body to be adjusted for a particular sterilization process. For certain embodiments, preferably the walls of the heat-transfer modulating body have a thermal diffusivity (a) of not more than 1×10⁻⁵ m²/s at 20° C.

The process challenge devices described herein can be provided without or with one or more process indicators. The indicator or indicators are chosen to be used with sterilization conditions to be employed in a particular sterilization process. When the device is provided without the indicator, the indicator is selected and placed in the device prior to using the device in the sterilization process. For example, for a steam sterilization process, a steam sterilization indicator is selected for the indicator. Moreover, the indicator can be chosen based upon the amount of exposure to sterilization conditions required to cause the indicator to indicate that the exposure has occurred. The choice of the sterilization indicator can thereby by used to increase or decrease the resistance of the sterilization process challenge device.

When absorbent material is present, the space within the container is dimensioned to allow the indicator and absorbent material to fit within the space. The absorbent material can absorb the condensate of a sterilant to prevent or reduce the amount of condensate that can contact the sterilization indicator, thereby preventing undesired indicator error. Furthermore, preventing condensate formation on the indicator reduces the heat gain of the indicator caused by heat transfer from the sterilant. For example, with steam sterilization, the absorbent material absorbs water which would otherwise condense on the indicator. A suitable absorbent material is cellulose or other absorbent fiber, such as absorbent paper.

The absorbent material can extend beyond the ends of the indicator, such that when placed within a space within a container, the indicator can be retrieved from the space by pulling on the absorbent material.

For certain embodiments, when an indicator is within a space within a container, the distance between indicator and the walls defining the space is preferably less than 5 cm. For certain embodiments the distance is less than 2 cm, 1 cm, 0.75 cm, or 0.5 cm. For certain embodiments, the indicator can contact the walls. Preferably, the distance between the indicator and the walls is sufficient to allow a layer of absorbent material between the walls and the indicator.

The process challenge device of the present invention can be provided without or with an indicator, which is chosen to be used with sterilization conditions to be employed in a particular sterilization process. As indicted above, the indicator can be a BI, a CI, a combination thereof. A plurality of indicators can also be used in the process challenge device. When the device is provided without an indicator, an indicator is selected and placed in the device prior to using the device in a sterilization process. The indicator or indicators can be covered with a porous material, such as paper or fabric. For certain embodiments, the indicator is sandwiched between or wrapped in two or more layers of a porous material. For certain embodiments, preferably the porous material absorbs sterilant condensate.

As indicated above, for certain embodiments, the space within the container contains a volume of gas of at least 5 cm³. For certain embodiments, including any one of the above embodiments of the device described herein, the volume of gas contained within the space is at least 10, 25, or 50 cm³. For certain of these embodiments, the volume of gas contained within the space is not more 1000 cm³, 500 cm³, 250 cm³, 125 cm³, or 75 cm³. Because the containers described herein include at least one flexible wall, the above volumes are typically determined at atmospheric pressure.

As indicated above, for certain embodiments, the walls comprising any one of the heat-transfer modulating bodies described herein have a thickness of at least 0.3 cm. For certain embodiments, including any one of the embodiments of the device described herein, the thickness is preferably at least about 0.5 cm. For certain of these embodiments, the thickness is at least 0.6 cm, 0.75 cm, 1 cm, 1.25 cm, or 2.5 cm. For certain embodiments, the thickness is at most 10 cm or 5 cm.

For certain embodiments, the walls comprising any one of the heat-transfer modulating bodies described herein have a thermal diffusivity (α) of not more than 1×10⁻⁵ m²/s at 20° C. Suitable materials for the walls of the body include, for example, stainless steel (α=0.405×10⁻⁵ m²/s), polypropylene, DELRIN, nylon (α=1.3×10⁻⁷ m²/s), polyester, polycarbonate, polytetrafluoroethylene (α=1.1×10⁻⁷ m²/s), and the like. For certain of these embodiments, the thermal diffusivity is not more than 5×10⁻⁷ M²/s at 20° C. For certain of these embodiments, the thermal diffusivity is not more than 2×10⁻⁷ m²/s at 20° C. The thermal diffusivity of the material indicates how rapidly the material adjusts its temperature to that of its surroundings. For example, a material with a relatively low thermal diffusivity heats up more slowly than a material with a higher thermal diffusivity in an environment at an elevated temperature, such as a sterilization chamber. Thermal diffusivity is used in heat transfer analysis and is the ratio of thermal conductivity to volumetric heat capacity as follows:

α=κ/ρCₚ

where κ is thermal conductivity (W/mK), ρ is density (kg/m³), and Cₚ is specific heat capacity (J/kgK). Thus, using these parameters, a suitable material or combination of materials for the walls of the heat-transfer modulating body can be chosen to achieve a desired resistance to sterilization conditions used in a sterilization process. For example, a material with a particular thermal diffusivity can be used for the walls of the heat-transfer modulating body, or the walls of the heat-transfer modulating body can be comprised of two or more layers, where at least two of the layers have different thermal diffusivities, to provide walls with a composite thermal diffusivity.

Sterilization indicators which can be used in the process challenge device described herein are known and include biological indicators and chemical indicators. Examples of biological indicators include ATTEST 1292 Rapid Biological Indicators (available from 3M Company, St. Paul, Minn.) and those described in U.S. Pat. No. 6,623,955 can be used. Examples of chemical indicators include COMPLY STERIGAGE 1243 Steam Chemical Integrator (available from 3M Company) and those described in U.S. Pat. No. 5,916,816 can be used.

The following is a list of certain exemplary embodiments of the present invention.

1. A process challenge device comprising:

a first container comprising walls which define a first space within the first container;

a process indicator within the first space;

at least one sterilant access for a sterilant to enter the first space within the first container;

wherein at least one of the walls which defines the first space is a flexible wall; and wherein the walls are impervious to the sterilant.

2. The device of embodiment 1, further comprising a heat-transfer modulating body adjacent the indicator.
3. The device of embodiment 2, wherein at least a portion of the body at least surrounds the indicator.
4. The device of embodiment 3, wherein the at least a portion of the heat-transfer modulating body comprises walls which surround the indicator; and wherein the walls are impervious to the sterilant.
5. The device of embodiment 3, wherein the at least a portion of the heat-transfer modulating body comprises walls which envelop the indicator; and wherein at least a portion of the walls enveloping the indicator is pervious to the sterilant.
6. The device of any one of embodiment 2 through 5, wherein the heat-transfer modulating body comprises walls having a thickness of at least 0.3 cm.
7. The device of any one of embodiments 1 through 6, wherein the first space has a volume which can vary as a pressure differential varies between the first space and outside of the first container.
8. The device of embodiment 7, wherein the volume can vary by at least 10 percent.
9. The device of embodiment 8, wherein the volume can vary by at least 50 percent.
10. The device of any one of embodiments 1 through 9, wherein the first space further contains a volume of gas at atmospheric pressure of at least 5 cubic centimeters.
11. The device of embodiment 10, wherein the volume of gas is not more than 1000 cubic centimeters.
12. The device of any one of embodiments 1 through 11, wherein the at least one sterilant access comprises at least one opening in at least one of the walls which defines the first space, wherein the at least one opening has an area.
13. The device of embodiment 12, wherein the at least one opening has an area of at least 0.5 mm².
14. The device of embodiment 12 or embodiment 13, wherein the at least one opening has an area of not more than 100 cm².
15. The device of any one of embodiments 12, 13, and 14, wherein the area of the at least one opening is maintained during a sterilization process.
16. The device of any one of embodiments 1 through 11, wherein the at least one sterilant access comprises at least two openings in at least one of the walls which defines the first space.
17. The device of embodiment 16, wherein the at least two openings are in at least two walls which define the first space.
18. The device of embodiment 17, wherein the at least two walls are on opposing sides of the first container.
19. The device of any one of embodiments 16, 17, and 18, wherein each of the at least two openings independently has an area of at least 0.5 mm².
20. The device of any one of embodiment 16 through 19, wherein each of the at least two openings independently has an area of not more than 50 cm².
21. The device of any one of embodiments 1 through 11, wherein the at least one sterilant access comprises at least one duct comprising at least two layers of sheet material, each having an area and at least one major surface adjacent to a major surface of another of the at least two layers, each major surface comprising at least a portion of the area.
22. The device of any one of embodiments 12 through 20, wherein the at least one sterilant access further comprises at least one duct comprising at least two layers of sheet material, each having an area and at least one major surface adjacent to a major surface of another of the at least two layers, each major surface comprising at least a portion of the area.
23. The device of embodiment 21 or embodiment 22, wherein a porous spacer is positioned between the at least one major surface and the major surface of another of the at least two layers.
24. The device of any one of embodiments 21, 22, and 23, wherein the at least one major surface and the major surface of another of the at least two layers are spaced apart by a plurality of raised areas on at least one of the major surfaces.
25. The device of any one of embodiments 21 through 24, wherein the major surfaces adjacent to each other have an area of at least 10 mm² where the major surfaces are adjacent to each other.
26. The device of any one of embodiments 21 through 25, wherein the major surfaces adjacent to each other have an area of not more than 100 cm² where the major surfaces are adjacent to each other.
27. The device of any one of embodiments 1 through 11, wherein the at least one sterilant access comprises at least one pressure actuating valve.
28. The device of any one of embodiments 12 through 26, wherein the at least one sterilant access further comprises at least one pressure-actuating valve.
29. The device of embodiment 27 or embodiment 28, wherein the at least one pressure-actuating valve is a combination valve.
30. The device of any one of embodiments 27, 28, and 29, wherein the at least one pressure-actuating valve is actuated when there is a pressure difference between the first space within the first container and outside of the first container.
31. The device of embodiment 30, wherein the pressure difference is at least 6.895 kPa (1 ps)i.
32. The device of embodiment 30 or embodiment 31, wherein the pressure difference is not more than 345 kPa (50 psi).
33. The device of any one of embodiments 1 through 11, wherein the sterilant access comprises at least two pressure-actuating valves.
34. The device of any one of embodiments 12 through 26, wherein the sterilant access further comprises at least two pressure-actuating valves.
35. The device of embodiment 33 or embodiment 34, wherein at least one pressure-actuating valve is actuated when the pressure is higher outside of the first container than in the first space within the first container.
36. The device of any one of embodiments 33, 34, and 35, wherein at least one pressure-actuating valve is actuated when the pressure is higher in the first space within the first container than outside of the first container.
37. The device of embodiment 35 or embodiment 36, wherein the pressure is higher by at least 6.895 kPa (1 psi).
38. The device of any one of embodiments 35, 36 and 37, wherein the pressure is higher by not more than 345 kPa (50 psi).
39. The device of any one of embodiments 1 through 38, further comprising a second container comprising:

walls which define a second space within the second container; and

at least one sterilant access for a sterilant to enter the second space;

wherein the first container is within the second space; wherein at least one of the walls which defines the second space is a flexible wall; and wherein the walls which define the second space are impervious to the sterilant.

40. The device of embodiment 39, wherein the second space has a volume which can vary as a pressure differential varies between the second space and outside of the second container.
41. The device of embodiment 40, wherein the volume can vary by at least 10 percent.
42. The device of embodiment 41, wherein the volume can vary by at least 50 percent.
43. The device of any one of embodiments 39 through 42, wherein the second space further contains a volume of gas at atmospheric pressure of at least 5 cubic centimeters.
44. The device of embodiment 43, wherein the volume of gas is not more than 1000 cubic centimeters.
45. The device of any one of embodiments 39 through 44, wherein the at least one sterilant access for a sterilant to enter the second space comprises at least one opening in at least one of the walls which defines the second space.
46. The device of embodiment 45, wherein the at least one opening has an area of at least 0.5 mm².
47. The device of embodiment 45 or embodiment 46, wherein the at least one opening has an area of not more than 100 cm².
48. The device of any one of embodiments 45, 46, and 47, wherein the area of the at least one opening is maintained during a sterilization process.
49. The device of any one of embodiments 39 through 44, wherein the at least one sterilant access for a sterilant to enter the second space comprises at least two openings in at least one of the walls which defines the second space.
50. The device of embodiment 49, wherein the at least two openings are in at least two walls which define the second space.
51. The device of embodiment 50, wherein the at least two walls are on opposing sides of the second container.
52. The device of any one of embodiments 49, 50, and 51, wherein each of the at least two openings independently has an area of at least 0.5 mm².
53. The device of any one of embodiment 49 through 52, wherein each of the at least two openings independently has an area of not more than 50 cm².
54. The device of any one of embodiments 39 through 44, wherein the at least one sterilant access for a sterilant to enter the second space comprises at least one duct comprising at least two layers of sheet material, each having at least one major surface adjacent to a major surface of another of the at least two layers.
55. The device of any one of embodiments 45 through 53, wherein the at least one sterilant access for a sterilant to enter the second space further comprises at least one duct comprising at least two layers of sheet material, each having at least one major surface adjacent to a major surface of another of the at least two layers.
56. The device of embodiment 54 or embodiment 55, wherein a porous spacer is positioned between the at least one major surface and the major surface of another of the at least two layers.
57. The device of any one of embodiments 54, 55, and 56, wherein the at least one major surface and the major surface of another of the at least two layers are spaced apart by a plurality of raised areas on at least one of the major surfaces.
58. The device of any one of embodiments 54 through 53, wherein the major surfaces adjacent to each other have an area of at least 10 mm² where the major surfaces are adjacent to each other.
59. The device of any one of embodiments 54 through 58, wherein the major surfaces adjacent to each other have an area of not more than 100 cm² where the major surfaces are adjacent to each other.
60. The device of any one of embodiments 39 through 44, wherein the at least one sterilant access for a sterilant to enter the second space comprises at least one pressure-actuating valve.
61. The device of any one of embodiments 45 through 59, wherein the at least one sterilant access for a sterilant to enter the second space further comprises at least one pressure-actuating valve.
62. The device of embodiment 60 or embodiment 61, wherein the at least one pressure-actuating valve is a combination valve.
63. The device of any one of embodiments 60, 61, and 62, wherein the at least one pressure-actuating valve is actuated when there is a pressure difference between the second space within the second container and outside of the second container.
64. The device of embodiment 63, wherein the pressure difference is at least 6.895 kPa (1 psi).
65. The device of embodiment 63 or embodiment 64, wherein the pressure difference is not more than 345 kPa (50 psi).
66. The device of any one of embodiments 39 through 44, wherein the sterilant access for a sterilant to enter the second space comprises at least two pressure-actuating valves.
67. The device of any one of embodiments 45 through 59, wherein the sterilant access for a sterilant to enter the second space further comprises at least two pressure actuating valves.
68. The device of embodiment 66 or embodiment 67, wherein at least one pressure-actuating valve is actuated when the pressure is higher outside of the second container than in the second space within the second container.
69. The device of any one of embodiments 66, 67, and 68, wherein at least one pressure-actuating valve is actuated when the pressure is higher in the second space within the second container than outside of the second container.
70. The device of embodiment 68 or embodiment 69, wherein the pressure is higher by at least 6.895 kPa (1 psi).
71. The device of any one of embodiments 68, 69 and 70, wherein the pressure is higher by not more than 345 kPa (50 psi).
72. The device of any one of embodiments 39 through 70, further comprising at least one additional container comprising:

walls which define at least one additional space within the at least one additional container; and

at least one sterilant access for a sterilant to enter the at least one additional space;

wherein the second container is within the at least one additional space; wherein at least one of the walls which defines the at least one additional space is a flexible wall; and wherein the walls which define the at least one additional space are impervious to the sterilant.

73. The device of embodiment 72, wherein the at least one additional space has a volume which can vary as a pressure differential varies between the at least one additional space and outside of the at least one additional container.
74. The device of embodiment 69, wherein the volume can vary by at least 10 percent.
75. The device of embodiment 74, wherein the volume can vary by at least 50 percent.
76. The device of any one of embodiments 72 through 75, wherein the at least one additional space further contains a volume of gas at atmospheric pressure of at least 5 cubic centimeters.
77. The device of embodiment 76, wherein the volume of gas is not more than 1000 cubic centimeters.
78. The device of any one of embodiments 72 through 77, wherein the at least one sterilant access for a sterilant to enter the at least one additional space comprises at least one opening in at least one of the walls which defines the at least one additional space, and wherein the opening has an area.
79. The device of embodiment 78, wherein the at least one opening has an area of at least 0.5 mm².
80. The device of embodiment 78 or embodiment 79, wherein the at least one opening has an area of not more than 100 cm².
81. The device of any one of embodiments 78, 79, and 80, wherein the area of the at least one opening is maintained during a sterilization process.
82. The device of any one of embodiments 72 through 77, wherein the at least one sterilant access for a sterilant to enter the at least one additional space comprises at least two openings in at least one of the walls which defines the at least one additional space.
83. The device of embodiment 82, wherein the at least two openings are in at least two walls which define the at least one additional space.
84. The device of embodiment 83, wherein the at least two walls are on opposing sides of the at least one additional container.
85. The device of any one of embodiments 82, 83, and 84, wherein each of the at least two openings independently has an area of at least 0.5 mm².
86. The device of any one of embodiment 82 through 85, wherein each of the at least two openings independently has an area of not more than 50 cm².
87. The device of any one of embodiments 72 through 77, wherein the at least one sterilant access for a sterilant to enter the at least one additional space comprises at least one duct comprising at least two layers of sheet material, each having at least one major surface adjacent to a major surface of another of the at least two layers.
88. The device of any one of embodiments 78 through 86, wherein the at least one sterilant access for a sterilant to enter the at least one additional space further comprises at least one duct comprising at least two layers of sheet material, each having at least one major surface adjacent to a major surface of another of the at least two layers.
89. The device of embodiment 87 or embodiment 88, wherein a porous spacer is positioned between the at least one major surface and the major surface of another of the at least two layers.
90. The device of any one of embodiments 87, 88, and 89, wherein the at least one major surface and the major surface of another of the at least two layers are spaced apart by a plurality of raised areas on at least one of the major surfaces.
91. The device of any one of embodiments 87 through 90, wherein the major surfaces adjacent to each other have an area of at least 10 mm² where the major surfaces are adjacent to each other.
92. The device of any one of embodiments 87 through 91, wherein the major surfaces adjacent to each other have an area of not more than 100 cm² where the major surfaces are adjacent to each other.
93. The device of any one of embodiments 72 through 77, wherein the at least one sterilant access for a sterilant to enter the at least one additional space comprises at least one pressure-actuating valve.
94. The device of any one of embodiments 78 through 92, wherein the at least one sterilant access for a sterilant to enter the at least one additional space further comprises at least one pressure-actuating valve.
95. The device of embodiment 93 or embodiment 94, wherein the at least one pressure-actuating valve is a combination valve.
96. The device of any one of embodiments 93, 94, and 95, wherein the at least one pressure-actuating valve is actuated when there is a pressure difference between the at least one additional space within the at least one additional container and outside of the at least one additional container.
97. The device of embodiment 96, wherein the pressure difference is at least 6.895 kPa (1 psi).
98. The device of embodiment 96 or embodiment 97, wherein the pressure difference is not more than 345 kPa (50 psi).
99. The device of any one of embodiments 72 through 77, wherein the sterilant access for a sterilant to enter the at least one additional space comprises at least two pressure actuating valves.
100. The device of any one of embodiments 78 through 92, wherein the sterilant access for a sterilant to enter the at least one additional space further comprises at least two pressure actuating valves.
101. The device of embodiment 99 or embodiment 100, wherein at least one pressure-actuating valve is actuated when the pressure is higher outside of the at least one additional container than in the at least one additional space within the at least one additional container.
102. The device of any one of embodiments 99, 100, and 101, wherein at least one pressure-actuating valve is actuated when the pressure is higher in the at least one additional space within the at least one additional container than outside of the at least one additional container.
103. The device of embodiment 101 or embodiment 102, wherein the pressure is higher by at least 6.895 kPa (1 psi).
104. The device of any one of embodiments 101, 102 and 103, wherein the pressure is higher by not more than 345 kPa (50 psi).
105. The device of any one of embodiments 1 through 38, wherein the at least one sterilant access for a sterilant to enter the first space within the first container is sealed with at least one removable seal.
106. The device of embodiment 26 as dependent on embodiment 23 as dependent on embodiment 22 as dependent on embodiment 14 as dependent on embodiment 12 as dependent on embodiment 7 as dependent on embodiment 5 as dependent on embodiment 1, wherein the at least one opening has an area of 10 cm² to 100 cm²; wherein the at least two layers of sheet material have a width and a length extending from an outer surface of the first container into the first space within the container; wherein the major surfaces adjacent to each other have an area of at least 10 cm² where the major surfaces are adjacent to each other, wherein the porous spacer has a width which is 15 percent to 30 percent of the width of the sheet material and a length extending from an outer surface of the first container and which is at least 75 percent of the length of the sheet material.
107. The device of embodiment 106, wherein the at least one opening is sealed with at least one removable seal.
108. The device of embodiment 106 or embodiment 107, wherein the sheet material is a paper.
109. The device of any one of embodiments 106, 107, and 108, wherein the porous spacer is a nonwoven fabric.
110. The device of any one of embodiments 106 through 109, wherein the porous spacer can be removed from the duct.
111. The device of any one of embodiments 106 through 110, wherein the volume of the first space can vary by at least 100 percent.
112. The device of any one of embodiments 105 through 111, wherein all of the walls of the first container are flexible walls.
113. The device of embodiment 112, wherein the flexible walls are a laminate film.
114. The device of any one of embodiments 12 through 15, 45 through 48, 78 through 81, 106 through 111, and 112 and 113 as dependent on any one of embodiments 105 as dependent on any one of embodiments 12 through 15, and 106 through 111, wherein the a least one opening is at least partially surrounded with a stiffening material.
115. The device of any one of embodiments 16 through 20, 49 through 53, and 82 through 86, wherein at least one of the at least two openings is at least partially surrounded with a stiffening material.
116. The device of any one of embodiments 12 through 15, 45 through 48, 78 through 81, and 106 through 114, wherein the at least one opening is covered with a permeable vent material.
117. The device of any one of embodiments 16 through 20, 49 through 53, 82 through 86, and 115, wherein at least one of the at least two openings is covered with a permeable vent material.
118. The device of any one of embodiments 39 through 71, 114 as dependent on any one of embodiments 45 through 48, 115 as dependent on any one of embodiments 49 through 53, 116 as dependent on any one of embodiments 45 through 48, and 117 as dependent on any one of embodiments 49 through 53, wherein the at least one sterilant access for a sterilant to enter the second space within the second container is sealed with at least one removable seal.
119. The device of any one of embodiments 72 through 104, 114 as dependent on any one of embodiments 78 through 81, 115 as dependent on any one of embodiments 82 through 86, 116 as dependent on any one of embodiments 78 through 81, and 117 as dependent on any one of embodiments 82 through 86, wherein the at least one sterilant access for a sterilant to enter the at least one additional space within the at least one additional container is sealed with at least one removable seal.
120. The device of any one of embodiments 1 through 119, further comprising an absorbent material which absorbs sterilant condensate within the first container.
121. The device of any one of embodiments 39 through 104; 114 as dependent on any one of embodiments 45 through 48 and 78 through 81; 115 as dependent on any one of embodiments 49 through 53 and 82 through 86; 116 as dependent on any one of embodiments 45 through 48, 78 through 81 and 114 as dependent on any one of embodiments 45 through 48 and 78 through 81; 117 as dependent on any one of embodiments 49 through 53, 82 through 86 and 115 as dependent on any one of embodiments 49 through 53 and 82 through 86; 118; and 120 as dependent on any one of embodiments 39 through 104, 114 as dependent on any one of embodiments 45 through 48 and 78 through 81, 115 as dependent on any one of embodiments 49 through 53 and 82 through 86, 116 as dependent on any one of embodiments 45 through 48, 78 through 81 and 114 as dependent on any one of embodiments 45 through 48 and 78 through 81, 117 as dependent on any one of embodiments 49 through 53, 82 through 86 and 115 as dependent on any one of embodiments 49 through 53 and 82 through 86, further comprising an absorbent material which absorbs sterilant condensate within the second container.
122. The device of any one of embodiments 72 through 104; 114 as dependent on any one of embodiments 78 through 81; 115 as dependent on any one of embodiments 82 through 86; 116 as dependent on any one of embodiments 78 through 81 and 114 as dependent on any one of embodiments 78 through 81; 117 as dependent on any one of embodiments 82 through 86, and 115 as dependent on any one of embodiments 82 through 86; 119; 120 as dependent on any one of embodiments 72 through 104, 114 as dependent on any one of embodiments 78 through 81, 115 as dependent on any one of embodiments 82 through 86, 116 as dependent on any one of embodiments 78 through 81 and 114 as dependent on any one of embodiments 78 through 81, 117 as dependent on any one of embodiments 82 through 86 and 115 as dependent on any one of embodiments 82 through 86; and 121 as dependent on any one of embodiments 72 through 104, 114 as dependent on any one of embodiments 78 through 81, 115 as dependent on any one of embodiments 82 through 86, 116 as dependent on any one of embodiments 78 through 81 and 114 as dependent on any one of embodiments 78 through 81, 117 as dependent on any one of embodiments 82 through 86 and 115 as dependent on any one of embodiments 82 through 86, further comprising an absorbent material which absorbs sterilant condensate within the at least one additional container.
123. The device of any one of embodiments 120, 121 and 122, wherein the absorbent material is a sheet material adjacent the at least one sterilant access.
124. The device of embodiment 123, wherein the sheet material has a thickness of at least 0.25 mm and not more than 10 mm.
125. A method of determining the effectiveness of a sterilization process, the method comprising:

providing a process challenge device of any one of embodiments 1 through 124;

positioning the process challenge device in a sterilization chamber;

exposing the process challenge device to a sterilant at an elevated temperature; and

determining whether or not the process indicator indicates that it has been exposed to sterilization process conditions effective for sterilizing an article.

126. A method of determining the effectiveness of a sterilization process, the method comprising:

providing a process challenge device of any one of embodiments 105; 107; 108 through 113; 114, 116, 120, 123, and 124 as dependent on embodiment 107; 118; 119; 120, 121, 123, and 124 as dependent on embodiment 118; and 120 through 124 as dependent on embodiment 119;

removing at least one of the at least one removable seal;

positioning the process challenge device in a sterilization chamber;

exposing the process challenge device to a sterilant at an elevated temperature; and

determining whether or not the process indicator indicates that it has been exposed to sterilization process conditions effective for sterilizing an article.

127. The method of embodiment 125 or embodiment 126, further comprising positioning the process challenge device in a volume restrictor which prevents the process challenge device from exceeding a pre-determined volume.
128. The method of any one of embodiments 125, 126, and 127, further comprising positioning the article in the sterilization chamber.
129. A kit comprising a plurality of process challenge devices of any one of embodiments 1 through 124.
130. A kit comprising a plurality of process challenge devices selected from the group consisting of a plurality of process challenge devices of any one of embodiments 1 through 38 and 105 through 113, a plurality of process challenge devices of any one of embodiments 39 through 71 and 106, a plurality of process challenge devices of any one of embodiments 72 through 104 and 107, and a combination thereof.
131. The kit of embodiment 129 or embodiment 130, wherein the first spaces within the first containers of the plurality of process challenge devices have the same volumes or different volumes.
132. The kit of any one of embodiments 129, 130, and 131, further comprising at least one volume restrictor which prevents the process challenge device from exceeding a pre-determined volume.

Objects and advantages of this invention are further illustrated by the following examples, but the particular materials and amounts thereof recited in these examples, as well as other conditions and details, should not be construed to unduly limit this invention.

### EXAMPLES

### Example 1

### Sterilant Access Ducts

The Attest™ Rapid 5 Test Pack Plus #41382 containing an Attest™ 1292 Rapid Biological Indicators (Attest BIs) and Comply™ SteriGage™ 1243 Steam Chemical Integrator (SteriGage), all available from 3M Company, St. Paul, Minn., was placed into a heat sealable poly-foil pouch to increase the time required to inactivate the biological and chemical indicators inside the test pack. The construction of the Attest Test Pack is shown in FIG. 2 in U.S. Pat. No. 4,636,472. The Attest Rapid 5 Test Packs are designed to be used to monitor a 4 minute 132° C. (270° F.) 4 pulse pre-vacuum sterilizer, so the indicators are inactivated within 4 minutes. The pouch CadPak N is available from TechniPac Inc, Le Sueur, Minn. The pouch is a multiple layer pouch consisting of nylon, polyethylene, foil and polyethylene layers. The pouch had a duct at one end to restrict air removal and steam penetration into the package as shown in FIG. 2. The duct was constructed of two pieces of 10 mil index paper, taped around the outside to maintain alignment of the edges and to create a gap between the two pieces of index paper as illustrated in FIG. 3. The ducts were approximately 14 cm long and widths of 1.27, 2.54 and 5.08 cm were evaluated for a surface area of 17.8, 35.6 and 71.1 cm² respectively. The Attest Rapid 5 Test Pack was wrapped with an absorbent towel to prevent the heat sealable layer inside the pouch from adhering to the test pack during sterilization. The paper towel is commercially available as Kleenex® Premiere®, Kimberly-Clark, Roswell, Ga. One end of the duct was inserted into the outer wrap on the bottom of the Attest Rapid 5 Test Pack. The other end extended outside the pouch and was heat sealed into the pouch creating an opening into the pack.

The ATTEST Rapid 5 Test Packs in the heat sealed pouches were exposed in a 132° C. (270° F.) 4 pulse pre-vacuum sterilizer, AMSCO® Eagle Model 3013 Sterilizer, Steris Corporation, Mentor, Ohio. The packs were exposed at 10, 15, 17.5 and 20 minutes. The vacuum and pressure pulse for each cycle used a vacuum level of 20 inches of Mercury (in Hg) and a pressure pulse of 239.2 kPa (20 psig (pounds per square inch gravity)). The Attest Rapid 5 Test Packs were exposed without the heat sealed pouches and exposed for 2 and 4 minutes at the same sterilization conditions.

After exposure, the SteriGage (SG) chemical integrators were read to determine if the moving front indicator dye had moved into the Reject or Accept region of the indicator. The Attest BIs were activated by crushing the inner ampules and incubating the indicators in the Attest Model 290 Autoreaders, 3M Company, St. Paul, Minn. which detects the fluorescence caused by the enzymatic breakdown of an enzyme substrate in the growth medium. After 3 hours of incubation, the Autoreader activates a green light indicating an acceptable sterilization cycle or a red light is activated to indicate a fluorescent positive indicator and a sterilization cycle failure. The indicators continued incubating for a total of 48 hours at 60° C. to allow surviving spores to grow and cause a visual color change in the growth medium from purple to yellow. The color change to yellow indicates a sterilization failure.

The results are shown in Table 1. The data shows the pouch pack significantly increased the exposure time required to inactivate the biological and chemical indicators inside the test pack. The Attest Rapid 5 Test Packs tested outside of the pouches had no positive BIs or CIs indicating a cycle failure (reject) after 2 and 4 minutes. The same test packs tested inside the pouches with the described ducts had all positive BIs and all the CIs indicating a sterilization failure after 10 minutes of exposure at the same conditions. This increased resistance would be useful for monitoring sterilization cycles of greater than 10 minutes.

TABLE 1

| | | | | |
|---|---|---|---|---|
| | Exposure | | Attest 1292 | Attest 1292 |
| | Time | | 3 Hour | 48 Hour |
| No. Tested | (minutes) | SteriGage | Fluorescence | Growth |

71.1 cm² Ducts No. Reject CIs or Positive BIs

| | | | | |
|---|---|---|---|---|
| 2 | 10 | 2 | 2 | 2 |
| 3 | 10 | 3 | 3 | 3 |
| 3 | 10 | 3 | 3 | 3 |
| 2 | 15 | 0 | 2 | 1 |
| 3 | 15 | 0 | 2 | 1 |
| 3 | 15 | 0 | 2 | 1 |
| 3 | 17.5 | 0 | 2 | 0 |
| 3 | 17.5 | 0 | 2 | 0 |
| 2 | 20 | 0 | 0 | 0 |
| 3 | 20 | 0 | 0 | 0 |
| 3 | 20 | 0 | 0 | 0 |

17.8 cm² Ducts No. Reject CIs or Positive BIs

| | | | | |
|---|---|---|---|---|
| 2 | 10 | 2 | 2 | 2 |
| 2 | 15 | 2 | 2 | 2 |
| 2 | 20 | 0 | 1 | 0 |

35.6 cm² Ducts No. Reject CIs or Positive BIs

| | | | | |
|---|---|---|---|---|
| 2 | 10 | 2 | 2 | 2 |
| 2 | 15 | 0 | 2 | 2 |
| 2 | 20 | 0 | 0 | 0 |

Attest Rapid 5 Outside the Pouch No. Reject CIs or Positive BIs

| | | | | |
|---|---|---|---|---|
| 3 | 2 | 0 | 0 | 0 |
| 3 | 4 | 0 | 0 | 0 |

### Example 2

### Sterilant Access Multi-Layer Duct

The same construction described in Example 1 was used in this example, except the ducts were modified to decrease the time to inactivate the biological and chemical indicators. In this example, the ducts were a multi-layer construction using two pieces of 10 mil index paper with a piece of cotton towel sandwiched between the index paper to increase the vent opening as shown in FIG. 4. The duct was held together with tape around the outside creating an opening between the two pieces of index paper and towel. The ducts were approximately 14 cm long and widths of 0.64 cm and 0.95 cm were evaluated for a surface area of 9 and 13.3 cm² respectively.

The results are shown in Table 2. The data shows the multi-layer vent construction can decrease the time to inactivate the biological and chemical indicators inside the test pack. This construction would be useful for monitoring sterilization cycles of 10 minutes.

TABLE 2

| | | | | |
|---|---|---|---|---|
| | Exposure | | Attest 1292 | Attest 1292 |
| | Time | | 3 Hour | 48 Hour |
| No. Tested | (minutes) | SteriGage | Fluorescence | Growth |

13.3 cm² Multi-Layer Ducts No. Reject CIs or Positive BIs

| | | | | |
|---|---|---|---|---|
| 3 | 5 | 1 | 2 | 1 |
| 3 | 5 | 0 | 3 | 3 |
| 3 | 5 | 1 | 2 | 2 |
| 3 | 7.5 | 0 | 0 | 0 |
| 3 | 7.5 | 0 | 2 | 1 |
| 3 | 7.5 | 0 | 0 | 0 |
| 3 | 10 | 0 | 0 | 0 |
| 3 | 10 | 0 | 0 | 0 |
| 3 | 10 | 0 | 1 | 0 |

9 cm² Multi-Layer Ducts No. Reject CIs or Positive BIs

| | | | | |
|---|---|---|---|---|
| 3 | 5 | 2 | 3 | 3 |
| 3 | 7.5 | 1 | 3 | 3 |
| 3 | 10 | 0 | 3 | 2 |

### Example 3

### Pouch with Small Diameter Openings

In this example, the Attest Rapid 5 Test Pack was heat sealed inside a clear polyester and polypropylene laminate film commercially available from Alcan Packaging as Material 123. The pouch was vented using 18 gauge needle to puncture holes into the film. Three different configurations were compared. Packs were tested with one hole on top of the pack as shown in FIG. 6, two holes with one on the top and one on the bottom of the pack, and one hole on top with a channel of film under the hole as shown in FIGS. 7A and 7B. The exposure conditions and the BIs and CIs were tested as described in Example 1. The packs were exposed for 5, 10, 15 and 20 minutes in a 132° C. pre-vacuum sterilizer.

The results in Table 3 shows the pouch pack with pin hole openings in the film significantly increased the exposure time required to inactivate the biological and chemical indicators inside the test pack. This increased resistance would be useful for monitoring sterilization cycles of 20 minutes.

TABLE 3

| | Exposure | | Attest 1292 | Attest 1292 |
|---|---|---|---|---|
| | Time | | 3 Hour | 48 Hour |
| Description | (minutes) | SteriGage | Fluorescence | Growth |
| Laminate Film | 5 | Reject | + | + |
| w/1 opening | | | | |
| Laminate Film | 5 | Reject | + | + |
| w/2 openings | | | | |
| Laminate Film | 5 | Reject | + | + |
| w/channel | | | | |
| opening | | | | |
| Laminate Film | 10 | Reject | + | + |
| w/1 opening | | | | |
| Laminate Film | 10 | Reject | + | + |
| w/2 openings | | | | |
| Laminate Film | 10 | Reject | + | + |
| w/channel | | | | |
| opening | | | | |
| Laminate Film | 15 | Accept | + | + |
| w/1 opening | | | | |
| Laminate Film | 15 | Accept | + | + |
| w/2 openings | | | | |
| Laminate Film | 15 | Accept | + | + |
| w/channel | | | | |
| opening | | | | |
| Laminate Film | 20 | Accept | + | − |
| w/1 opening | | | | |
| Laminate Film | 20 | Accept | − | − |
| w/2 openings | | | | |
| Laminate Film | 20 | Accept | − | − |
| w/channel | | | | |
| opening | | | | |

### Example 4

### Sterilant Access Openings for 121° C. Gravity Sterilization Process

In this example, the Attest Rapid 5 Test Packs were sealed in the film-foil pouch described in Example 1. The pouch was vented by cutting a 5.08 by 7.62 cm opening in the pack. Packs were made with one opening on the top for an opening area of 38.7 cm² as shown in FIG. 8 or with one opening on the top and bottom of the packs for an opening area of 77.4 cm². The packs were exposed in 121° C. gravity displacement sterilization cycles. Attest Rapid 5 Test Packs that were not sealed inside the film-foil packs were tested to compare to the packaged test packs.

The data in Table 4 shows the BIs and CIs in the unwrapped Attest Rapid 5 Test Packs were inactivated after 20 minutes of exposure at 121° C. The data in Table 5 shows the packs with one die cut opening on the top of the packs were not inactivated after 30 minutes of exposure. The packs with die cut openings on the top and bottom of the pack had fluorescent positive results after 25 minutes and all kill after 30 minutes of exposure. This example demonstrates how film-foil pouch can increase time required to inactivate the sterilization indicators inside the Attest Rapid 5 Test Packs.

TABLE 4

| | Exposure | | Attest 1292 | Attest 1292 |
|---|---|---|---|---|
| | Time | | 3 Hour | 48 Hour |
| Description | (minutes) | SteriGage | Fluorescence | Growth |
| Attest Test Pack | 10 | Reject | + | + |
| Attest Test Pack | 10 | Reject | + | + |
| Attest Test Pack | 15 | Reject | + | + |
| Attest Test Pack | 15 | Reject | + | + |
| Attest Test Pack | 20 | Accept | − | − |
| Attest Test Pack | 20 | Accept | − | − |

TABLE 5

| | Exposure | | Attest 1292 | Attest 1292 |
|---|---|---|---|---|
| | Time | | 3 Hour | 48 Hour |
| Description | (minutes) | SteriGage | Fluorescence | Growth |
| 2 openings top | 20 | Reject | + | + |
| and bottom | | | | |
| 2 openings top | 20 | Reject | + | + |
| and bottom | | | | |
| 1 opening top | 20 | Reject | + | + |
| 1 opening top | 20 | Reject | + | + |
| 2 openings top | 25 | Accept | + | − |
| and bottom | | | | |
| 2 openings top | 25 | Accept | + | − |
| and bottom | | | | |
| 1 opening top | 25 | Reject | + | + |
| 1 opening top | 25 | Reject | + | + |
| 2 openings top | 30 | Accept | − | − |
| and bottom | | | | |
| 2 openings top | 30 | Accept | − | − |
| and bottom | | | | |
| 1 opening top | 30 | Reject | + | + |
| 1 opening top | 30 | Reject | + | + |
| 2 openings top | 40 | Accept | − | − |
| and bottom | | | | |
| 2 openings top | 40 | Accept | − | − |
| and bottom | | | | |
| 1 opening top | 40 | Accept | − | − |
| 1 opening top | 40 | Accept | + | − |

### Example 5

### Double Pouch

In this example, the Attest Rapid 5 Test Pack was heat sealed inside a clear polyester and polypropylene laminate film commercially available from Alcan Packaging as Material 123. The pouch had a 1.27 cm diameter hole in the bottom of the pack. This pouch was heat sealed inside another pouch, which had a 1.27 cm diameter hole on the top side of the pack for an opening area of 1.27 cm². The double pouch design is shown in FIGS. 9A and 9B. The exposure conditions and the BIs and CIs were tested as described in Example 1. The packs were exposed for 5, 10, 15 and 20 minutes in a 132° C. pre-vacuum sterilizer.

The results in Table 6 shows the double pouch pack significantly increased the exposure time required to inactivate the biological and chemical indicators inside the test pack. This increased resistance would be useful for monitoring sterilization cycles of 20 minutes or longer.

TABLE 6

| | Exposure | | | |
|---|---|---|---|---|
| | Time | | 3 hr | 48 hr |
| | (minutes) | SG | Flu. | G |
| Double Pouch | 5 | Reject | + | + |
| Double Pouch | 10 | Reject | + | + |
| Double Pouch | 15 | Reject | + | + |
| Double Pouch | 20 | Accept | + | − |

### Example 6

### BI and CI Heat Sealed Inside a Pouch with Sterilant Access Opening

In this example, Attest™ 1292 Rapid Biological Indicators (Attest BIs) and Comply™ SteriGage™ 1243 Steam Chemical Integrator (SteriGage or SG), all available from 3M Company, St. Paul, Minn., was wrapped in an absorbent paper towel and placed into a heat sealable laminate film commercially available from Clear Lam Packaging, Elk Grove Village, Ill., as 50/0015 PP. An opening was provided in the top of the film using an 18 gauge needle to pierce the film. This design is illustrated in FIG. 1. The exposure conditions and the BIs and CIs were tested as described in Example 1. The packs were exposed for 5, 10, 15 and 20 minutes in a 132° C. pre-vacuum sterilizer.

The results in Table 7 shows the BIs and CIs inside the pouch pack significantly increased the exposure time required 10 minutes of exposure to be inactivated. This increased resistance would be useful for monitoring sterilization cycles of greater than 5 minutes.

TABLE 7

| | Exposure | | | |
|---|---|---|---|---|
| | Time | | 3 hr | 48 hr |
| | (minutes) | SG | Flu. | G |
| BI/CI in Pouch | 5 | Reject | + | + |
| BI/CI in Pouch | 10 | Accept | − | − |
| BI/CI in Pouch | 15 | Accept | − | − |
| BI/CI in Pouch | 20 | Accept | − | − |

### Example 7

### BIs and CIs Inside a Polypropylene Housing and Sealed Inside a Pouch with Sterilant Access Opening

In this example, Attest™ 1292 Rapid Biological Indicators (Attest BIs) and Comply™ SteriGage™ 1243 Steam Chemical Integrator (SteriGage or SG), all available from 3M Company, St. Paul, Minn., was wrapped in an absorbent paper towel and placed into a polypropylene body (as described in FIG. 1A of U.S. Patent Application No. 61/050,513) which was heat sealable inside a laminate film commercially available from Clear Lam Packaging, Elk Grove Village, Ill., as 50/0015 PP. An opening was provided in the top of the film using an 18 gauge needle to pierce the film. This design is illustrated in FIG. 10. The exposure conditions and the BIs and CIs were tested as described in Example 1. The packs were exposed for 5, 10, 15 and 20 minutes in a 132° C. pre-vacuum sterilizer.

The results in Table 8 show the BIs and CIs inside the polypropylene insert and pouch pack significantly increased the exposure time needed to inactivate the BIs and CIs. This increased resistance would be useful for monitoring sterilization cycles of greater than 20 minutes.

TABLE 8

| | Exposure | | | |
|---|---|---|---|---|
| | Time | | 3 hr | 48 hr |
| | (minutes) | SG | Flu. | G |
| BI/CI in Polypropylene Body | 5 | Reject | + | + |
| BI/CI in Polypropylene Body | 10 | Reject | + | + |
| BI/CI in Polypropylene Body | 15 | Reject | + | + |
| BI/CI in Polypropylene Body | 20 | Reject | + | + |

### Example 8

### BI and CI Sealed in a Pouch with Sterilant Access and Place into a Heat-Transfer Modulating Body

In this example, Attest™ 1292 Rapid Biological Indicators (Attest BIs) and Comply™ SteriGage™ 1243 Steam Chemical Integrator (SteriGage or SG), all available from 3M Company, St. Paul, Minn., was wrapped in an absorbent paper towel and heat sealable inside a laminate film commercially available from Clear Lam Packaging, Elk Grove Village, Ill., as 50/0015 PP. An opening was provided in the top of the film using an 18 gauge needle to pierce the film. The BI and CI package was then inserted into a polypropylene body. This body was as illustrated for body 520 in FIG. 10. However, unlike FIG. 10, the laminate film container (with the BI and CI) was positioned within the body where indicator 580 is shown in FIG. 10. The exposure conditions and the BIs and CIs were tested as described in Example 1. The packs were exposed for 5, 10, 15 and 20 minutes in a 132° C. pre-vacuum sterilizer.

The results in Table 9 show the BIs and CIs inside the pouch and polypropylene heat sink were inactivated in 5 minutes. This design would be useful for monitoring sterilization cycles of less than 5 minutes.

TABLE 9

| | Exposure | | | |
|---|---|---|---|---|
| | Time | | 3 hr | 48 hr |
| | (minutes) | SG | Flu. | G |
| BI/CI in Pouch in Polypropylene | 5 | Accept | − | − |
| Body | | | | |
| BI/CI in Pouch in Polypropylene | 10 | Accept | − | − |
| Body | | | | |
| BI/CI in Pouch in Polypropylene | 15 | Accept | − | − |
| Body | | | | |
| BI/CI in Pouch in Polypropylene | 20 | Accept | − | − |
| Body | | | | |

### Example 9

### Pouch with Short Ducts for Sterilant Access

A 5.08 cm wide duct was constructed as described in Example 1 except the length was reduced to 2.54 cm for a surface area of 12.9 cm². The Clear Lam packaging described in Example 6 was used to package the Attest Rapid 5 Test Pack described in Example 1. The exposure conditions and the BIs and CIs were tested as described in Example 1. The packs were exposed for 5, 10, 15 and 20 minutes in a 132° C. pre-vacuum sterilizer.

TABLE 10

| | Exposure | | | |
|---|---|---|---|---|
| | Time | | 3 hr | 48 hr |
| | (minutes) | SG | Flu. | G |
| Pouch with 12.9 cm² Ducts | 5 | Reject | + | + |
| Pouch with 12.9 cm² Ducts | 10 | Accept | − | − |
| Pouch with 12.9 cm² Ducts | 15 | Accept | + | + |
| Pouch with 12.9 cm² Ducts | 20 | Accept | − | − |

### Example 10

### General Construction, Sterilization, and Analysis of Process Challenge Device with Enclosed Test Pack

Materials from an ATTEST Rapid 5 Steam Plus Test Pack (3M Company, Saint Paul, Minn.) were used to construct a test pack according to U.S. Pat. No. 4,636,472, containing an ATTEST 1292 Rapid Biological Indicator (Attest BI) and COMPLY STERIGAGE 1243 Steam Chemical Integrator (Comply SteriGage), both available from 3M Company, Saint Paul, Minn.

A pouch sized to accommodate this test pack was created by heat sealing two opposing sheets of clear polyester and polypropylene laminate film, commercially available as PERFECFLEX 35881-E (Perfecseal Incorporated, Oshkosh, Wis.) or Material Code 123 (Alcan Packaging, Rio Tinto Alcan, Montreal, Quebec, Canada). Next, a circular hole of specified diameter (pouch vent) was punched out from a specified location of one side of the clear film pouch.

The test pack was inserted into the clear film pouch, such that the top side (label side, with outer laminate index card) was facing toward the punched hole of the clear film pouch. Next, a sheet of absorbent material was placed within the clear film pouch between the top side of the test pack and pouch vent of the clear film pouch. The open end of the clear film pouch was then heat sealed shut, creating a test pack in a closed pouch with a single pouch vent.

The pouch-enclosed test pack was exposed in a 132° C. (270° F.) 4 pulse pre-vacuum cycle for a specified exposure time using an AMSCO Eagle Model 3013-C sterilizer (Steris Corporation, Mentor, Ohio). The vacuum and pressure pulse set points were 24 inches of mercury vacuum and 280.6 kilopascal (26 pounds per square inch gauge), respectively.

After the sterilization cycle, the pouch-enclosed test pack was disassembled to remove the contained Attest BI and Comply SteriGage.

For the indicators, the Comply SteriGage was measured to determine the length of the moving front indicator dye using a Mitutoyo ABSOLUTE DIGIMATIC digital caliper (Mitutoyo Corporation, Kawasaki, Kanagawa, Japan). The length was measured from the starting edge of the device to the closest point of the indicator dye moving front. A longer moving indicator dye length indicated increased exposure to the sterilant. The Attest BI was activated by crushing the inner ampoule and incubating the indicator in an ATTEST Model 290 Autoreader (3M Company, St. Paul, Minn.), which detected fluorescence emitted by the enzymatic breakdown of an enzyme substrate in the growth medium. After 3 hours of incubation, the Autoreader activated either a green light indicating negligible fluorescence change and an acceptable sterilization cycle or a red light indicating significant fluorescence change and a sterilization cycle failure. After the rapid fluorescence readout, the indicator was incubated at 60° C. for a total of 48 hours to allow any surviving spores to multiply causing a pH indicator-mediated visual color change in the growth medium from purple to yellow. After growth incubation, the growth medium color purple indicated an acceptable sterilization cycle, while color change to yellow indicated a sterilization cycle failure.

### Example 11

### Process Challenge Device with Enclosed Test Pack with Absorbent Material

PCDs containing test packs with enclosed Attest BI and Comply SteriGage were constructed with pouches as described in Example 10. Pouches had a 1.27 cm (0.5 inch) diameter pouch vent in a central location 3.81 cm (1.5 inches) from the end of the test pack opposite the sealed end of the pouch. See FIG. 17. Process challenge devices (PCDs) were constructed as pouch-enclosed test packs with and without a 10.16 cm (4 inch) by 15.24 (6 inch) sheet of Kimberly-Clark KOTEX LIGHTDAYS Absorbent #714847 (Kimberly-Clark Corporation, Roswell, Ga.) as shown in FIG. 17.

The PCDs were exposed in a 132° C. 4-pulse pre-vacuum cycle for 4 minutes as described in Example 10. These cycle conditions are generally accepted to yield adequate sterilization.

After sterilization, the PCDs were disassembled and the enclosed Attest BI and Comply SteriGage were analyzed for fluorescence readout and growth color change, and moving front indicator dye length, respectively.

The data in Table 11 show that an absorbent material adjusted the resistance of the PCD to provide sufficient but not excessive resistance under adequate sterilization conditions.

TABLE 11
Attest BI and Comply SteriGage results for pouch-enclosed
test packs without and with absorbent material in a 132°
C. 4-pulse pre-vacuum cycle for 4 minutes exposure.

| | | | Comply SteriGage |
|---|---|---|---|
| | | | Indicator Dye Length |
| Absorbent | Attest BI | Attest BI | Average +/− Standard |
| Material | Fluorescence | Growth | Deviation (mm) |
| No Absorbent | Positive | Positive | 25.68 +/− 3.31 |
| Material | Positive | Positive | |
| | Positive | Positive | |
| Kimberly-Clark | Negative | Negative | 37.91 +/− 0.47 |
| COTEX | Negative | Negative | |
| LIGHTDAYS | Negative | Negative | |
| Absorbent | | | |

### Example 12

### Pouch-Enclosed Test Pack with Different Absorbent Materials

PCDs containing test packs with enclosed Attest BI and Comply SteriGage were constructed with pouches as described in Example 10. Pouches had a 1.27 cm diameter pouch opening in a central location 3.81 cm from the end of the test pack opposite the sealed end of the pouch and with and without a 10.16 cm by 15.24 cm sheet of 3M Chemical Sorbent P-110 (3M Company, Saint Paul, Minn.) or Kimberly-Clark KOTEX LIGHTDAYS Absorbent #714847 (Kimberly-Clark Corporation, Roswell, Ga.).

The pouch-enclosed test packs were exposed in a 132° C. 4-pulse pre-vacuum cycle for 4 minutes as described in Example 10. These conditions are considered to be an adequate sterilization cycle.

After sterilization, the pouch-enclosed test packs were disassembled and the enclosed Attest BI and Comply SteriGage were analyzed for fluorescence readout and growth color change, and moving front indicator dye length, respectively.

The data in Table 12 show that type of absorbent material in the pouch-enclosed test pack construction may be used to adjust overall pouch-enclosed test pack resistance.

TABLE 12
Attest BI and Comply SteriGage results for pouch-enclosed
test packs with various absorbent materials in a 132°
C. 4-pulse pre-vacuum cycle for 4 minutes exposure.

| | | | Comply SteriGage |
|---|---|---|---|
| | | | Indicator Dye Length |
| Absorbent | Attest BI | Attest BI | Average +/− Standard |
| Material | Fluorescence | Growth | Deviation (mm) |
| 3M Chemical | Positive | Positive | 33.07 +/− 0.24 |
| Sorbent P-110 | Negative | Negative | |
| | Positive | Negative | |
| Kimberly-Clark | Negative | Negative | 37.91 +/− 0.47 |
| COTEX | Negative | Negative | |
| LIGHTDAYS | Negative | Negative | |
| Absorbent | | | |

### Example 13

### Pouch-Enclosed Test Pack with Varying Absorbent Material Thickness

PCDs containing test packs with enclosed Attest BI and Comply SteriGage were constructed with pouches as described in Example 10. Pouches had a 1.90 cm (0.75 inch) diameter pouch vent in a central location 3.81 cm from the end of the test pack opposite the sealed end of the pouch and different numbers of stacked 10.16 cm by 15.24 cm 3M Chemical Sorbent P-110 sheets.

The pouch-enclosed test packs were exposed in a 132° C. 4-pulse pre-vacuum cycle for 2 minutes as described in Example 10.

After sterilization, the pouch-enclosed test packs were disassembled and the enclosed Attest BI and Comply SteriGage were analyzed for fluorescence readout and growth color change, and moving front indicator dye length, respectively.

The data in Table 13 show that increased thickness of absorbent material in a pouch-enclosed test pack construction decreases overall pouch-enclosed test pack resistance.

TABLE 13
Attest BI and Comply SteriGage results for pouch-enclosed
test packs with varying absorbent sheet thickness in a 132°
C. 4-pulse pre-vacuum cycle for 2 minutes exposure.

| | | | Comply SteriGage |
|---|---|---|---|
| | | | Indicator Dye Length |
| Absorbent | Attest BI | Attest BI | Average +/− Standard |
| Thickness | Fluorescence | Growth | Deviation (mm) |
| 1 3M P-110 Sheet | Positive | Positive | 25.58 +/− 1.34 |
| | Positive | Positive | |
| | Positive | Positive | |
| 2 3M P-110 | Negative | Negative | 30.09 +/− 1.40 |
| Sheets | Negative | Negative | |
| | Negative | Negative | |

### Example 14

### Pouch-Enclosed Test Pack with Varying Absorbent Lateral Dimensions

PCDs containing test packs with enclosed Attest BI and Comply SteriGage were constructed with pouches as described in Example 10. Pouches had a 1.90 cm diameter pouch vent in a central location 3.81 cm from the end of the test pack opposite the sealed end of the pouch and 2.54 cm 1 inch) diameter circle, 10.16 cm (4 inch) by 5.08 cm (2 inch) sheet, or 10.16 cm by 15.24 cm sheet of 3M Chemical Sorbent P-110 placed directly below the pouch vent.

The pouch-enclosed test packs were exposed in a 132° C. 4-pulse pre-vacuum cycle for 4 minutes as described in Example 10. These conditions are considered to be an adequate sterilization cycle.

After sterilization, the pouch-enclosed test packs were disassembled and the enclosed Attest BI and Comply SteriGage were analyzed for fluorescence readout and growth color change, and moving front indicator dye length, respectively.

The data in Table 14 show that a range of lateral dimensions of absorbent material in a pouch-enclosed test pack yields similar overall pouch-enclosed test pack resistance, although there may be a minimum set of lateral dimensions which may be used to attain a desired overall pouch-enclosed test pack resistance under adequate sterilization conditions.

TABLE 14
Attest BI and Comply SteriGage results for pouch-enclosed
test packs with varying absorbent sheet thickness in a 132°
C. 4-pulse pre-vacuum cycle for 4 minutes exposure.

| | | | Comply SteriGage |
|---|---|---|---|
| | | | Indicator Dye Length |
| Absorbent | Attest BI | Attest BI | Average +/− Standard |
| Dimension | Fluorescence | Growth | Deviation (mm) |
| 2.54 cm diameter | Positive | Positive | 27.54 +/− 2.40 |
| circle | Positive | Positive | |
| | Positive | Positive | |
| 10.16 cm by 5.08 | Negative | Negative | 36.32 +/− 0.93 |
| cm sheet | Negative | Negative | |
| | Negative | Negative | |
| 10.16 cm by | Negative | Negative | 37.84 +/− 1.17 |
| 15.24 cm sheet | Negative | Negative | |
| | Negative | Negative | |

### Example 15

### Pouch-Enclosed Test Pack with Different Vent Diameters

PCDs containing test packs with enclosed Attest BI and Comply SteriGage were constructed with pouches as described in Example 10. A 10.16 cm by 15.24 cm sheet of 3M Chemical Sorbent P-110 and a pouch vent diameter of 1.17 cm (0.46 inch) or 2.29 cm (0.90 inch) in a central location 3.81 cm from the end of the test pack opposite the sealed end of the pouch were used.

The pouch-enclosed test packs were exposed in a 132° C. 4-pulse pre-vacuum cycle for 4 minutes as described in Example 10.

After sterilization, the pouch-enclosed test packs were disassembled and the enclosed Attest BI and Comply SteriGage were analyzed for fluorescence readout and growth color change, and moving front indicator dye length, respectively.

The data in Table 15 shows that increased pouch vent diameter in the pouch-enclosed test pack construction decreased overall pouch-enclosed test pack resistance.

TABLE 15
Attest BI and Comply SteriGage results for pouch-enclosed
test packs with varying pouch vent diameters in a 132°
C. 4-pulse pre-vacuum cycle for 4 minutes exposure.

| | | | Comply SteriGage |
|---|---|---|---|
| | | | Indicator Dye Length |
| | Attest BI | Attest BI | Average +/− Standard |
| Vent Diameter | Fluorescence | Growth | Deviation (mm) |
| 1.17 cm | Positive | Positive | 31.47 +/− 1.06 |
| | Positive | Negative | |
| | Positive | Positive | |
| 2.29 cm | Negative | Negative | 35.91 +/− 0.32 |
| | Negative | Negative | |
| | Negative | Negative | |

### Example 16

### Pouch-Enclosed Test Pack with Different Pouch Vent Locations

PCDs containing test packs with enclosed Attest BI and Comply SteriGage were constructed with pouches as described in Example 10. A 10.16 cm by 15.24 cm 3M Chemical Sorbent P-110 sheet and pouch vent diameter of 1.90 cm in a central location either 3.81 cm or 7.62 cm (3 inches) from the end of the test pack opposite the sealed end of the pouch were used.

The pouch-enclosed test packs were exposed in a 132° C. 4-pulse pre-vacuum cycle for 4 minutes as described in Example 10.

After sterilization, the pouch-enclosed test packs were disassembled and the enclosed Attest BI and Comply SteriGage were analyzed for fluorescence readout and growth color change, and moving front indicator dye length, respectively.

The data in Table 16 show that the increased pouch vent distance from the end of the test pack opposite the sealed end of the pouch in pouch-enclosed test pack construction increased overall pouch-enclosed test pack resistance.

TABLE 16
Attest BI and Comply SteriGage results for pouch-enclosed
test packs with varying pouch vent locations in a 132°
C. 4-pulse pre-vacuum cycle for 4 minutes exposure.

| Distance of Pouch | | | |
|---|---|---|---|
| Vent from End of | | | Comply SteriGage |
| Test Pack Opposite | | | Indicator Dye Length |
| the Sealed End | Attest BI | Attest BI | Average +/− Standard |
| of the Pouch | Fluorescence | Growth | Deviation (mm) |
| 3.81 cm | Negative | Negative | 36.17 +/− 2.81 |
| | Negative | Negative | |
| | Negative | Negative | |
| 7.62 cm | Positive | Positive | 28.38 +/− 1.58 |
| | Positive | Positive | |
| | Positive | Positive | |

### Example 17

### Pouch-Enclosed Test Pack with Different Number of Index Cards in Test Packs

PCDs containing test packs with enclosed Attest BI and Comply SteriGage were constructed with pouches as described in Example 10 except that the number of index cards in the test packs was varied. A 10.16 cm by 15.24 cm 3M Chemical Sorbent P-110 sheet, a 1.90 cm diameter pouch vent in a central location 3.81 cm from the end of the test pack opposite the sealed end of the pouch, and varying numbers of die-cut index cards, specifically 74 or 104 die-cut cards in the test packs were used.

The pouch-enclosed test packs were exposed in a 132° C. 4-pulse pre-vacuum cycle for 3.5 minutes as described in Example 10.

After sterilization, the pouch-enclosed test packs were disassembled and the enclosed Attest BI and Comply SteriGage were analyzed for fluorescence readout and growth color change, and moving front indicator dye length, respectively.

The data in Table 17 show that the increased number of index cards in a pouch-enclosed test pack construction increased overall pouch-enclosed test pack resistance.

TABLE 17
Attest BI and Comply SteriGage results for pouch-enclosed
test packs with varying number of die-cut index cards in a
132° C. 4-pulse pre-vacuum cycle for 3.5 minutes exposure.

| | | | Comply SteriGage |
|---|---|---|---|
| Number of | | | Indicator Dye Length |
| Die-Cut | Attest BI | Attest BI | Average +/− Standard |
| Index Cards | Fluorescence | Growth | Deviation (mm) |
| 74 | Negative | Negative | 33.30 +/− 0.68 |
| | Negative | Negative | |
| | Negative | Negative | |
| 104 | Positive | Positive | 29.24 +/− 2.14 |
| | Positive | Positive | |
| | Negative | Negative | |

### Example 18

### Pouch-Enclosed Test Pack with Absorbent and Vent Material

PCDs containing test packs with enclosed Attest BI and Comply SteriGage were constructed with pouches as described in Example 10. Pouches had a single 0.559 cm (0.22 inch) diameter pouch vent in a central location 3.81 cm from the end of the test pack opposite the sealed end of the pouch and a 10.16 by 15.24 cm 3M Chemical Sorbent P-110 sheet.

Additionally, a circular swatch of Fiberweb TYPAR 3801 (a polypropylene spunbond nonwoven available from Fiberweb, Old Hickory, Tenn.) pouch vent material was adhered onto the pouch covering the pouch vent using a circular piece of pressure sensitive adhesive backed aluminum foil. See FIG. 18.

The addition of pouch vent material further limits sterilant entry into the pouch-enclosed test pack and, therefore, increases overall pouch-enclosed test pack resistance.

### Example 19

### Different Pouch-Enclosed Test Packs with Comparable Resistance

PCDs containing test packs with enclosed Attest BI and Comply SteriGage were constructed with pouches as described in Example 10. Specifically, either a 10.16 cm by 15.24 cm 3M Chemical Sorbent P-110 sheet and 1.90 cm pouch vent or a 10.16 cm by 15.24 cm Kimberly-Clark KOTEX LIGHTDAYS Absorbent #714847 sheet and 1.27 cm pouch vent in a central location 3.81 cm from the end of the test pack opposite the sealed end of the pouch were used.

The pouch-enclosed test packs were exposed in a 132° C. 4-pulse pre-vacuum cycle for 4 minutes as described in Example 10.

After sterilization, the pouch-enclosed test packs were disassembled and the enclosed Attest BI and Comply SteriGage were analyzed for fluorescence readout and growth color change, and moving front indicator dye length, respectively.

The data in Table 18 show absorbent material and pouch vent diameter parameters can be adjusted such that different pouch-enclosed test pack constructions yield comparable overall pouch-enclosed test pack resistance.

TABLE 18
Attest BI and Comply SteriGage results for pouch-enclosed
test packs with varying construction in a 132°
C. 4-pulse pre-vacuum cycle for 4 minutes exposure.

| | | | Comply SteriGage |
|---|---|---|---|
| Pouch-Enclosed | | | Indicator Dye Length |
| Test Pack | Attest BI | Attest BI | Average +/− Standard |
| Construction | Fluorescence | Growth | Deviation (mm) |
| 3M Chemic Sorbent | Negative | Negative | 37.84 +/− 1.17 |
| P-110/ | Negative | Negative | |
| 1.90 cm pouch vent | Negative | Negative | |
| Kimberly-Clark | Negative | Negative | 37.91 +/− 0.47 |
| COTEX | Negative | Negative | |
| LIGHTDAYS | Negative | Negative | |
| Absorbent/1.27 cm | | | |
| pouch vent | | | |

### Example 20

### Pouch-Enclosed Test Pack with Absorbent Material and Rigid (Stiffening) Material around the Pouch Vent

PCDs containing test packs with enclosed Attest BI and Comply SteriGage were constructed with pouches as described in Example 10. Specifically, a 10.16 cm by 15.24 cm 3M Chemical Sorbent P-110 sheet was used.

Additionally, a square adhesive mailing label Avery White Mailing Label #5360 (Avery Dennison Corporation, Pasadena, Calif.) was adhered over the area to be punched out as the pouch vent. Then, a single 0.838 cm (0.33 inch) diameter pouch vent was punched out through the pouch and adhesive label in a central location 3.81 cm from the end of the test pack opposite the sealed end.

The rigid material around the pouch vent provides additional support and aids in preventing pouch wrinkling at the point of the pouch vent. The rigid material improves pouch vent seating onto the absorbent material upon pressurization of the sterilization cycle and, therefore, produces more reproducible overall pouch-enclosed test pack resistance.

All references and publications or portions thereof cited herein are expressly incorporated herein by reference in their entirety into this disclosure. Exemplary embodiments of this invention are discussed and reference has been made to some possible variations within the scope of this invention. These and other variations and modifications in the invention will be apparent to those skilled in the art without departing from the scope of the invention, and it should be understood that this invention is not limited to the exemplary embodiments set forth herein. Accordingly, the invention is to be limited only by the embs provided below and equivalents thereof.

1. A process challenge device comprising:
a first container comprising walls which define a first space within the first container;
a process indicator within the first space;
at least one sterilant access for a sterilant to enter the first space within the first container;
wherein at least one of the walls which defines the first space is a flexible wall; and
wherein the walls are impervious to the sterilant.

2. The device of claim 1, further comprising a heat-transfer modulating body adjacent the indicator.

3. The device of claim 2, wherein at least a portion of the body at least surrounds the indicator.

4. (canceled)

5. The device of claim 1, wherein the first space has a volume which can vary as a pressure differential varies between the first space and outside of the first container.

6. The device of claim 1, wherein the first space further contains a volume of gas at atmospheric pressure of at least 5 cubic centimeters.

7. The device of claim 1, wherein the at least one sterilant access comprises at least one opening in at least one of the walls which defines the first space, wherein the at least one opening has an area.

8. (canceled)

9. The device of claim 1, wherein the at least one sterilant access comprises at least one duct comprising at least two layers of sheet material, each having an area and at least one major surface adjacent to a major surface of another of the at least two layers, each major surface comprising at least a portion of the area.

10. (canceled)

11. The device of claim 9, wherein a porous spacer is positioned between the at least one major surface and the major surface of another of the at least two layers.

12. (canceled)

13. The device of claim 1, wherein the at least one sterilant access comprises at least one pressure actuating valve.

14. (canceled)

15. The device of claim 1, further comprising a second container comprising:
walls which define a second space within the second container; and
at least one sterilant access for a sterilant to enter the second space;
wherein the first container is within the second space; wherein at least one of the walls which defines the second space is a flexible wall; and wherein the walls which define the second space are impervious to the sterilant.

16. The device of claim 15, wherein the second space has a volume which can vary as a pressure differential varies between the second space and outside of the second container.

17-22. (canceled)

23. The device of claim 1, further comprising a heat-transfer modulating body adjacent the indicator;
wherein at least a portion of the body at least surrounds the indicator;
wherein the at least a portion of the heat-transfer modulating body comprises walls which envelop the indicator; and wherein at least a portion of the walls enveloping the indicator is pervious to the sterilant;
wherein the first space has a volume which can vary as a pressure differential varies between the first space and outside of the first container;
wherein the at least one sterilant access comprises at least one opening in at least one of the walls which defines the first space, wherein the at least one opening has an area
wherein the at least one opening has an area of not more than 100 cm²;
wherein the at least one sterilant access further comprises at least one duct comprising at least two layers of sheet material, each having an area and at least one major surface adjacent to a major surface of another of the at least two layers, each major surface comprising at least a portion of the area;
wherein a porous spacer is positioned between the at least one major surface and the major surface of another of the at least two layers;
wherein the major surfaces adjacent to each other have an area of not more than 100 cm² where the major surfaces are adjacent to each other;
wherein the at least one opening has an area of 10 cm² to 100 cm²; wherein the at least two layers of sheet material have a width and a length extending from an outer surface of the first container into the first space within the container; wherein the major surfaces adjacent to each other have an area of at least 10 cm² where the major surfaces are adjacent to each other, wherein the porous spacer has a width which is 15 percent to 30 percent of the width of the sheet material and a length extending from an outer surface of the first container and which is at least 75 percent of the length of the sheet material.

24. The device of claim 7, wherein the a least one opening is at least partially surrounded with a stiffening material.

25. The device of claim 7, wherein the at least one opening is covered with a permeable vent material.

26. The device of claim 1, further comprising an absorbent material which absorbs sterilant condensate within the first container.

27. The device of claim 15, further comprising an absorbent material which absorbs sterilant condensate within the second container.

28. (canceled)

29. The device of claim 26, wherein the absorbent material is a sheet material adjacent the at least one sterilant access.

30. The device of claim 29, wherein the sheet material has a thickness of at least 0.25 mm and not more than 10 mm.

31. A method of determining the effectiveness of a sterilization process, the method comprising:
providing a process challenge device of claim 1;
positioning the process challenge device in a sterilization chamber;
exposing the process challenge device to a sterilant at an elevated temperature; and
determining whether or not the process indicator indicates that it has been exposed to sterilization process conditions effective for sterilizing an article.

32. A kit comprising a plurality of process challenge devices of claim 1.
